(19) United States
(12) Patent Application Publication
(10) Pub. No.: US 2024/0328314 A1
(43) Pub. Date: Oct. 3, 2024

(54) KINETIC MODELING OF PETROLEUM EVOLUTION

(71) Applicant: ConocoPhillips Company, Houston, TX (US)

(72) Inventor: Yishu SONG, Houston, TX (US)

(73) Assignee: ConocoPhillips Company, Houston, TX (US)

(21) Appl. No.: 18/591,418

(22) Filed: Feb. 29, 2024

Related U.S. Application Data
(60) Provisional application No. 63/492,363, filed on Mar. 27, 2023.

(51) Int. Cl. E21B 49/08; G01N 33/24; G01N 33/28

(52) U.S. Cl. CPC E21B 49/0875; E21B 49/088; G01N 33/241; G01N 33/2823

(57) ABSTRACT

A kinetic modeling method to describe/model the petroleum fluid evolution with respect to its bulk compositions and the isotope compositions. The bulk compositions are detailed to individual n-alkanes, while the isotope compositions are detailed to individual isotopomer within in each isotopologue of a given n-alkane. This provides a systematic solution to assess fluid maturity and to elucidate the charge history of a reservoir, based on the distribution of n-alkanes and detailed isotope composition of each n-alkanes.

### PRIOR RELATED APPLICATIONS

This application claims priority to U.S. Ser. No. 63/492,363, filed on Mar. 27, 2023, and incorporated by reference in its entirety for all purposes.

### FEDERALLY SPONSORED RESEARCH STATEMENT

Not applicable.

### FIELD OF THE DISCLOSURE

The disclosed methods relate generally to the exploration and production of petroleum from one or a multitude of reservoirs, by performing geochemical analyses and modeling to delineate and predict petroleum compositions and properties.

### BACKGROUND OF THE DISCLOSURE

Geochemical fingerprinting is a rapidly expanding discipline, anchored in the recognition that geological processes leave behind unique chemical and isotopic patterns in the rock record. In conventional oil fields, variations in petroleum composition may arise from one or a combination of source maturity, source facies variation, oil biodegradation, water washing, as well as charging and mixing of oils from different source rocks or of different maturity. In heavy oils and oil sands reservoirs, large-scale vertical and small-scale lateral variations in oil composition and fluid properties developed via interaction of biodegradation and charge mixing, resulting in orders of magnitude variation in viscosity over the thickness of the reservoir and large molecular variations within single wells.

Many of these patterns—informally referred to as “geochemical fingerprints”—differ only in fine detail from each other. For this reason, fingerprinting requires analytical data of very high precision and accuracy, and geochemical fingerprinting is by necessity an extremely data intensive process. Thus, significant work is needed to improve data collection and analysis, as well to develop new applications of this powerful technology.

U.S. Pat. Nos. 10,400,596 and 10,415,379 describe the use of multiply substituted isotopologue and position specific isotopes in geochemical fingerprinting. Isotopologues are molecules that differ only in their isotopic composition. They have the same chemical formula and bonding arrangement of atoms, but at least one atom has a different number of neutrons than the parent. One example is water, where its hydrogen-related isotopologues are: H₂O, HDO, D₂O, T₂O, HTO and DTO, where D is deuterium (2H) and T is tritium (3H). Position specific isotopes or site-specific isotope analysis is a branch of isotope analysis aimed at determining the isotopic composition of a particular atom position in a molecule.

For many molecules, when the population of all co-existing isotopologues reaches thermodynamic equilibrium, the clumped isotopologues are more abundant than one would expect for a random distribution of isotopes, and this excess is generally controlled by temperature. It is therefore possible to use clumped isotopes as “geothermometers,” as described in U.S. Pat. Nos. 10,400,596 and 10,415,379. The actual methods of data analysis were not provided in these patents, but a paper by the inventors (Peterson 2018) makes it clear that they used a Monte Carlo method. This is a stochastic method wherein they randomly substitute one carbon. However, the Monte Carlo simulation does not solve for time, so their method can only link isotope composition to transformation ratio (or percentage of conversion), not to the actual thermal maturity of a sample. Further, the method can only handle a single ¹³C substitution per alkane, which is inadequate for highly precise analysis. Thus, although an important advance in geochemistry, the data has not been optimized for accuracy, especially for low probability events.

This application provides more powerful data analysis of isotopologues, clumped isotopologues, and/or position specific isotopes in geochemical fingerprinting. The method thus improves data accuracy in a variety of applications, such as estimating the maturity of the source rock, determining the source facies from which the hydrocarbons were generated (e.g., marine or terrestrial source rocks), differentiating between potential origins of hydrocarbons (e.g., biogenic as compared to thermogenic), providing information on how much alteration hydrocarbons have experienced (e.g., from either biodegradation or thermal cracking), optimizing production, leak detection and mitigation, allocation of production, developing maturation and migration models for emerging plays, and the like.

### SUMMARY OF THE DISCLOSURE

The method involves obtaining a plurality of samples from a number of wells in one or a multitude of reservoirs across a geological region of interest and over a period of time and assigning both a time identifier and a location identifier to each of said plurality of samples.

Each of the plurality of samples is chemically fingerprinted to obtain bulk compositions, typically by whole oil gas chromatography (GC) analysis, as well as carbon isotope compositions, typically by gas chromatography isotope ratio mass spectrometry (GC-IRMS) analysis. Isotope compositions include but are not limited to isotope signature of different petroleum fractions (like saturates vs. aromatics), compound specific isotopes of C1 up to C40 (or higher as long as analytically amendable), isotopologue specific isotope compositions of light hydrocarbons (C1-C9), isotopomer specific isotope compositions of light hydrocarbons, and position specific isotope composition of light hydrocarbons.

Gas isotope ratio mass spectrometry is a fast-advancing analytical technique, which enables measuring of isotopic signatures with high precision and sensitivity thus revealing the history and origin of compounds in petroleum fluids. One suitable instrument setup for the isotope composition analyses is THERMO FISHER SCIENTIFIC'S™ MAT 253 sector mass spectrometer or their new ORBITRAP™ high resolution mass spectrometer coupled to gas chromatography.

To leverage the isotopic composition data and to better understand the evolution and/or charge history of petroleum fluids in a given reservoir, a novel kinetics model is developed herein to describe the bulk and isotope composition changes of petroleum fluids in a given reservoir over a given geological history and attempt to calibrate these modelled signatures with the measured bulk and isotope signatures obtained via geochemical analyses described above.

The kinetic model devised in this invention involves systematic kinetic descriptions of C—C bond cleavage upon reaction based on the properties of the reacting molecule (e.g., carbon chain length, dodecane vs. hexane), location of the C—C bond (e.g., terminal vs. internal), the isotope composition of carbon atom at each end of the C—C bond (e.g., ¹²C-¹²C vs. ¹²C-¹³C, ¹³C-¹³C), and isotope composition of the carbon atoms (if any) immediately next to the C—C bond.

One key feature of this kinetic model is its ability to model multiple ¹³C substitutions in a given hydrocarbon molecule with many isotopic isomers, instead of drastically simplifying the system by assuming only a single random ¹³C substitution per molecule, as was done with all of the prior art kinetic models.

The essence of kinetics modeling is to describe the changes over time. The model can explicitly calculate the isotope composition at any time point under any arbitrary temperature history. Vitrinite reflectance (% R_{O}) is a typical and more conventional expression of maturity, which itself is kinetically modeled/calculated, but it is less powerful than the new method developed herein. The methods discussed in U.S. Pat. No. 10,400,596 or U.S. Pat. No. 10,415,379 have limited capability to actually model time and maturity because they are based on how much reactant has converted to products (known as percentage of conversion, or transformation ratio), and is not explicitly or implicitly reaction time based.

Ultimately, this information can be used in any of the applications described above, but one application of particular interest to us is to identify the source of light end hydrocarbons in a reservoir, generated in-site via secondary cracking of early oil charge versus received as additional gas charge later in the reservoir history. In general, this is assessed by comparing the measured bulk composition and isotope compositions of reservoir fluids with kinetic modeling results. Secondary cracking products can be nicely modeled with the kinetics model regarding its bulk compositions and isotope compositions. Upon secondary cracking, the bulk composition changes and isotope composition changes are intertwined, allowing assessing stage of secondary cracking based on the bulk and isotope compositions of the petroleum fluid in question and the bulk and isotope compositions of the initial oil charge.

Later gassy charge, even from the same source bed that generated the original fluids in the reservoir prior to the onset of in-reservoir secondary cracking, can be modeled with a kinetic model of different initial setting (kerogen composition and thermal history—temperature and time), and the generated light hydrocarbons have different isotope signatures than those from in-reservoir cracking. Mixing of light hydrocarbons by these two sources will lead to isotope signatures different to each end member case (in-reservoir cracking vs. later gas charge), and the extent of mixing can be assessed based on the modeled end member bulk and isotope compositions. Worth mentioning again is that this kinetic model is capable—by design—of modeling the concentration change of individual isotopic isomers over time (e.g., for ethane ¹²CH₃¹²CH₃ vs. ¹²CH₃¹³CH₃ vs. ¹³CH₃¹³CH₃). This allows us to investigate the origin and/or the charge history of petroleum fluids in a given reservoir at deeper level with finer definition.

The data generated hereunder may of course be combined and integrated with traditional geochemical techniques, such as molecular (e.g., methane, ethane, carbon dioxide, nitrogen), bulk (e.g., mixtures of gases), stable isotope geochemistry (e.g., carbon, hydrogen, nitrogen, sulfur) of hydrocarbon and non-hydrocarbon gases, molecular geochemistry of oils (e.g., saturate and aromatic compounds), physical measurements (e.g., pressure, volume, and temperature (PVT)), and results from engineering tests and analyses (e.g., production logging test (PLT), pressure transient analysis, or decline curve analysis).

It may also be combined with bulk oil property measurements, such as boiling point distribution, density or API gravity, specific gravity, viscosity, pour point, cloud point, oxidation stability, sulfur content, BTU analysis, and the like.

Additional data that can be used with isotopic fingerprints include characterizations of kerogen and petroleum fluids (oil and gas) as follows:
TOC and Rock-Eval analyses of rock samples
Isolate kerogen from rock sample, and analyze the elemental composition (CHNOS) of kerogen isolate
Analyze the bulk carbon isotope composition of the kerogen isolate

For oil-either produced or extracted from rock with solvent—the following analyses are typical:
Whole oil GC analysis
SARA analysis
Bulk carbon isotope composition of the saturate and aromatics fractions
Compound specific isotope analysis (CSIA) of the oil (carbon isotope)

For gas-either produced gas, mud gas obtained during drilling, or gas retained in rock sample (core or cutting)—the following analyses are common:
Bulk gas composition by GC
Compound specific isotope analysis (carbon and hydrogen isotopes)

The analysis results will either be used as model input or calibration data and the geochemical analysis results and/or modeling results can then be used in a variety of applications, such as optimizing a reservoir production plan, tracing the source of various fluids, estimating the maturity of various fluids, mapping the reservoir, and the like.

The combination of geochemical analyses and kinetic modeling described in this invention will enhance existing technical capabilities for petroleum composition and property delineation and predictions, reservoir charge history analysis, development and production optimization, production performance prediction and production problem solving.

For example, using these technologies one can discriminate between production dominated by fracture flow and that dominated by free phase hydrocarbons or desorption of hydrocarbons from mineral or organic surfaces. Absorption and desorption of hydrocarbon to and from surfaces incur isotope fractionation. Typically, for the same hydrocarbon, say methane or ethane, the absorbed molecules will be isotopically heavier than free molecules (those not absorbed on to mineral or kerogen surfaces). Production stage dominated by hydrocarbons liberated into fractures upon fracking will have an isotope signature equivalent to a mixture of free hydrocarbon and absorbed hydrocarbon. Production stage dominated by rock matrix contribution will see different isotope signatures and it may change over time. Kinetic modeling of the isotope compositions of the fluids in reservoir is the foundation for such assessment.

In addition, contributions of hydrocarbons originating from multiple reservoir formations can be identified and quantified. For example, when artificial fractures penetrate through sealing strata and into other hydrocarbon bearing formations, or where artificial fractures intersect with natural fractures that result in contributions from other reservoirs, then the sample signatures will differ from the modeled signatures, indicating charging from these other sources.

Furthermore, these technologies may be used to constrain the source of hydrocarbons within well bores when wellbore integrity is under investigation or in aquifers when present at unusually high concentrations and contamination is suspected. This is a classic forensic application of geochemistry. Hydrocarbons from different sources have different isotope signatures, and the kinetics method can be used to model/predict the isotope signature of different sources and enhance the forensic investigation.

The technology therefore provides a mechanism to alter engineering practices and production strategies to maximize the volume and quality of hydrocarbon ultimately produced and also to mitigate any issues associated with the production of hydrocarbons if this is shown to be resulting in the leakage of hydrocarbons in the near well bore area.

The present methods include any of the following embodiments in any combination(s) of one or more thereof:

A method of estimating thermal maturity of hydrocarbon reservoir samples, comprising:
a) obtaining a plurality of samples from a hydrocarbon reservoir over a period of time and
assigning time and location identifiers to each of said plurality of samples;
b) fingerprinting each of said plurality of samples to obtain sample geochemical signatures
including sample bulk composition and sample carbon isotope signatures of one or more alkanes;
c) comparing said sample signatures to modeled signatures prepared by kinetic modeling to
provide an estimate of thermal maturity of each of said plurality of samples, wherein said kinetic
modeling comprises:
i) providing a stoichiometry matrix for a reaction network that describes each step in
cracking said one or more alkanes to methane, including all ¹²C and ¹³C isomers of all
reactants and all products, wherein only a single bond breaks in each step of said reaction
network;
ii) solving coupled differential equations to determine concentration changes of said all
reactants and said all products using an Arrhenius equation and reaction rate constant (k)
for each step in said stoichiometry matrix over time to obtain a concentration of all alkanes
in said reaction network at a specified heating rate and initial temperature, using an initial
concentration and a statistical abundance of each isomer; and
iii) summing concentrations of each isomer of each alkane and using said sums to provide
said modelled signatures.
A kinetic modeling method that comprises:
i) providing a stoichiometry matrix for a reaction network that describes each step in cracking
said one or more alkanes to methane, including all ¹²C and ¹³C isotope isomers of all reactants
and all products, wherein only a single bond breaks in each step of said reaction network;
ii) solving coupled differential equations to determine concentration changes of said all reactants
and said all products using an Arrhenius equation to calculate a reaction rate constant (k) for each
step in said stoichiometry matrix over time to obtain a concentration of all alkanes in said reaction
network at a specified heating rate and initial temperature, using an initial concentration and a
statistical and/or thermodynamic equilibrium abundance of each isomer; and
iii) summing concentrations of each isotope isomer of each alkane, and using said sums to
provide said modelled compositional signatures.
A kinetic method of modeling petroleum, comprising:
Step 1) constructing a set of reactions describing an evolution of petroleum fluids;
Step 2) permuting all isotopic isomers for each species in the set of reactions constructed in step 1,
replacing each species with its permutation of isotopic isomers, then building out a reaction networks
via permuting each reactant within the set of reactions constructed in step 1;
Step 3) building a stoichiometry matrix for said reaction network constructed in step 2;
Step 4) calculating a rate constant with an Arrhenius equation for each reaction built out in step 2 in
which a frequency factor and activation energy are a function of molecule size (number of carbon
atoms), position of the C—C bond to be broken, type of carbon (¹²C vs. ¹³C) of each carbon in the C—C
bond, and type of carbon next to each end of the C—C bond (if any);
Step 5) building a differential equation system for said reaction network built in step 2, with stoichiometry
built in step 3 and rate constants calculated in step 4;
Step 6) defining an initial condition for the differential equation system built in step 4, which entails
estimating a relative abundance of isotopic isomers via thermodynamic calculation (if attainable) or
statistic calculation (as a surrogate) based on a lumped carbon isotope signature for all isomers (as
known as compound specific δ¹³C value for a given alkane) and thermodynamic settings;
Step 7) defining a thermal history (temperature gradient and time span) corresponding to a desired
maturity (like % R_{O}) change;
Step 8) solving said differential equation system built in step 5 with initial conditions defined in step 6
over the thermal history defined in step 7, to generate a concentration of each species in said reaction
networks built in step 2 at each time step over the time span defined in step 7;
Step 9) preparing a model by summing all isotopic isomers of each alkane to obtain a petroleum bulk
composition, and calculating a compound specific isotope signature based on a relative abundance of
isotopic isomers and a number of ¹³C substitution and calculating ratios of isotopologues and/or
isotopomers for each alkane;
Step 10) calibrating (or comparing) said model of step 9 with available geochemical analysis results
from samples from a reservoir and refining steps 1-9 as needed to produce a final model; and
Step 11) using said final model from step 10 to optimize an exploration and/or production strategy.
-A method of optimizing hydrocarbon production, said method comprising: modeling hydrocarbon bulk
compositions and isotope compositions, wherein said bulk compositions are detailed to individual n-
alkanes, and said isotope compositions are detailed to individual isotopomer within in each isotopologue
of a given n-alkane; and using said modeling to optimize hydrocarbon production.
Any method herein described, wherein said carbon isotope signatures are selected from isotope
signature of different petroleum fractions (like saturates vs. aromatics), compound specific isotopes of
C1 up to C40 (or higher as long as analytically amendable), isotopologue specific isotope compositions
of light hydrocarbons, and position specific isotope composition of light hydrocarbons.
Any method herein described, wherein said fingerprinting step uses isotope ratio mass spectrometry
(IRMS), high resolution gas chromatography (HRGC); gas chromatography (GC); 2D gas
chromatography (GCxGC), mass spectrometry (MS); GC-MS; Fourier Transform Ion Cyclotron
Resonance MS (FTICR-MS); thin layer chromatography (TLC); two dimensional TLC (2D TLC); capillary
electrophoresis (CE); high pressure liquid chromatography (HPLC); Fourier Transform Infra-Red (FTIR)
spectrophotometry; X-ray fluorescence (XRF); atomic absorbance spectrophotometry (AAS);
Inductively Coupled Plasma MS (ICP-MS); Ion Chromatography (IC); nuclear magnetic resonance
(NMR); 2D GC-time of flight MS (GCxGC-TOFMS); saturate, aromatic, resin, and asphaltene levels
(SARA levels); carbon, hydrogen, nitrogen, oxygen and sulfur analysis (CHNOS analysis); elemental
analysis; GC isotope ratio MS (GC/IR-MS); or combinations thereof.
Any method herein described, said samples selected from one or more of core samples; cutting
samples; produced oil, water or gas samples; fractions of produced oil, water or gas samples; drilling
mud samples; or mud gas samples.
Any method herein described, wherein said location includes depth and lateral placement (x, y and z
axes).
Any method herein described, further comprising comparing said sample signatures with said modelled
signatures, wherein consistency of said sample signatures with said modelled signatures indicates
secondary cracking of in situ oil to form said samples, but deviations of said sample signatures from
said modelled signatures indicate charging of said samples from another source.
Any method herein described, said method further comprising mapping said reservoir according to said
estimated thermal maturities and said time and location identifiers, and deciding a well placement plan
based on said mapping.
Any method herein described, further comprising:
a) using said estimated thermal maturity in a reservoir model to predict production levels;
b) optimizing a production plan based on said predicted production levels; and
c) implementing said optimized production plan to produce hydrocarbons from said reservoir.
Any method herein described, wherein said reservoir model and said production plan includes one or
more of well placement, well depth, well arrangement, well completion, reservoir fracturing, reservoir
stimulation, enhanced oil production techniques, and combinations thereof.
Any method herein described, further comprising implementing said exploration and/or production
strategy to drill oil well(s) in said reservoir.
Any method herein described, further comprising implementing said exploration and/or production
strategy to produce hydrocarbons from said reservoir.
Any method herein described, further comprising implementing said exploration and/or production
strategy to produce hydrocarbons from said oil well(s).
Any method herein described, wherein said petroleum fluids include compounds from C1 to C40 or
higher.
Any method herein described, wherein said isotopic isomers include carbon isotopes ¹²C and ¹³C.

As used herein a “fingerprint” is an analysis of the bulk and/or isotopic compositions of a sample and is typically complex enough to uniquely identify the source of oil and gas samples. “Fingerprinting” refers to the analyses needed to generate the fingerprints. The results from fingerprinting may be called a “fingerprint” or a “signature.”

As used herein, the term “isotope” refers to one of two or more atoms with the same atomic number but with different numbers of neutrons. “Isotopologues” are molecules that differ only in their isotopic composition. Within an individual isotopologue, there can be multiple “isotopomers” which differ only in positions of isotope substitution. Isotope compositions are typically analyzed by isotope ratio mass spectrometry (IRMS), isotope ratio laser spectroscopy (IRLS), and nuclear magnetic resonance (NMR). When we discuss “isotope signatures” herein, we mean to also include all possible isotopologues and all isotopomers. The current work is exemplified with carbon isotopes, but in the future may be expanded to include hydrogen, oxygen, sulfur and nitrogen isotopes.

As used herein, “position-specific isotope analysis,” also called “site-specific isotope analysis,” is a branch of isotope analysis aimed at determining the isotopic composition of a particular atom position in a molecule. The distribution of isotopes across a molecule is often not stochastic, as the different isotopes will have different bond strength and can fractionate differently during chemical reactions.

As used herein “δ¹³C” or “delta ¹³C” is defined as: δ=1000 (R/R_{ref}−1), wherein R is the ratio of ¹³C/¹²C. R_{ref} refers to the standard to which the isotopic composition of the sample is compared.

As used herein, “gas wetness” is the ratio of the sum of ethane, propane, i-butane, n-butane, i-pentane and n-pentane to the sum of methane, ethane, propane, i-butane, n-butane, i-pentane and n-pentane (e.g., dry gas is mostly methane). Gas wetness decreases with increasing thermal maturity as the larger molecules crack to smaller ones, like methane.

As used herein, the term “geochemical signatures” or “geochemical fingerprints” refers to the chemical composition and/or chemical property characteristics of one or a multitude of samples under study. Such characteristics include but not limited to bulk properties like GOR, API gravity and gas wetness, bulk compositions like whole oil GC compositions, GCMS compositions of selected petroleum fraction, saturate and aromatic biomarkers, light hydrocarbon ratios, isotope compositions of one or a group of selected species, position specific isotope compositions, and clumped isotope compositions.

As used herein, the “Arrhenius equation” gives the dependence of the rate constant of a chemical reaction on the absolute temperature as k=Ae^{-Ea/RT}, where k is the rate constant, T is the absolute temperature (in Kelvin), A is the pre-exponential factor. Arrhenius originally considered A to be a temperature-independent constant for each chemical reaction. However, more recent treatments include some temperature dependence. Eₐ is the activation energy for the reaction (in the same units as RT), R is the universal gas constant.

Alternatively, the equation may be expressed as k=Ae^{-Ea/k}^{_{b}}^{T}, where Eₐ is the activation energy for the reaction (in the same units as k_{B}T), k_{B} is the Boltzmann constant. The only difference from the prior expression for the Arrhenius equation is the energy units of Eₐ: the former form uses energy per mole, which is common in chemistry, while the latter form uses energy per molecule directly, which is common in physics. The different units are accounted for in using either the gas constant, R, or the Boltzmann constant, k_{B}, as the multiplier of temperature T, but these two expressions are interchangeable.

As used herein, “deterministic kinetic modeling” refers to a process that entails constructing a system of coupled differential equations to describe the compositional evolution of petroleum fluids and solving these differential equations with a given initial condition over time.

As used herein, “Chung's plot” is plot of the isotopic composition of methane, ethane, propane, n-butane, n-pentane, etc. as a function of the reciprocal of the carbon number of the species. A linear trend supports a cogenetic origin (e.g., generated from the same source rock), whereas a non-linear fit suggests that the gas accumulation is a mixture of gases, a chemically altered gas, or a gas derived from a structurally heterogeneous carbon source. This plot is commonly known as a “Natural Gas Plot” or a “Chung Plot.”

As used herein, “R_{O}” is a measure of the percentage of incident light reflected from the surface of vitrinite particles in a sedimentary rock. It is referred to as % R_{O} (sometimes also written as R_{O}%). Results are often presented as a mean R_{O} value based on all vitrinite particles measured in an individual sample. % R_{O} is commonly used as source rock and petroleum fluid maturity indicator, and can be modeled with kinetic models over a given thermal history (temperature and time). Oil and gas generation boundaries can be established using vitrinite reflectance data. The boundaries are approximate and vary according to kerogen types.

As used herein, a “biomarker” in chemistry and geology are any suite of complex organic compounds composed of carbon, hydrogen and other elements or heteroatoms such as oxygen, nitrogen and sulfur, that are found in crude oils, bitumen, petroleum source rock and eventually show simplification in molecular structure from the parent organic molecules found in all living organisms. Essentially, they are complex carbon-based molecules derived from formerly living organisms. Each biomarker is quite distinctive when compared to its counterparts, as the time required for organic matter to convert to crude oil is discreet. Most biomarkers also usually have high molecular mass.

Some examples of biomarkers found in petroleum are pristane, triterpanes, steranes, phytane and porphyrin. Such petroleum biomarkers are produced via chemical synthesis using biochemical compounds as their main constituents. For instance, triterpanes are derived from biochemical compounds found on land angiosperm plants. The presence of petroleum biomarkers in small amounts in its reservoir or source rock make it necessary to use sensitive and differential approaches to analyze the presence of those compounds. The techniques typically used include gas chromatography and mass spectrometry.

The distribution of biomarker isomers can not only serve as fingerprints for oil/oil and oil/source correlation (to relate the source and reservoir) but also give geochemical information on organic source input (marine, lacustrine or land-based sources), age, maturity, depositional environment (for example, siliciclastic or carbonate, oxygen levels, salinity) and alteration (for example, water washing, biodegradation).

As used herein, a “reservoir” is a formation or a portion of a formation that includes sufficient permeability and porosity to hold and transmit fluids, such as hydrocarbons, water, natural gas, and the like.

A reservoir can have a plurality of chemically distinct “zones” therein, particularly in very tight rock, where mixing is almost non-existent. The data herein can be catalogued by zone, allowing that portion of the data to be used for other zones, even in other wells, as long as the zone has similar fingerprints.

As used herein, the term “hydrocarbons” refer to molecules formed primarily by hydrogen and carbon atoms, but may also include other elements, such as, but not limited to, halogens, metallic elements, nitrogen, oxygen, sulfur and the like. Hydrocarbons may be produced from petroleum reservoirs through wells penetrating a petroleum containing formation. Hydrocarbons derived from a petroleum reservoir have a wide variety of compositions, which are typically divided into different fractions, and which may include, but are not limited to, asphaltenes, resins, aromatics, saturates, natural gas liquid, dry gas, or combinations thereof.

“Petroleum fluids” (oil and gas) consist primarily of hydrocarbons, but may also contain non-hydrocarbon compounds, such as hydrogen sulfide (H₂S), carbon dioxide (CO₂), nitrogen (N₂), helium (He), etc.

As used herein, “hydrocarbon production” refers to any activity associated with extracting hydrocarbons from a well or other opening after the well is completed and production has been initiated. Accordingly, hydrocarbon production or extraction includes not only primary hydrocarbon extraction but also secondary and tertiary production techniques, such as injection of gas or liquid for increasing drive pressure, mobilizing the hydrocarbon or treating by, for example chemicals or hydraulic fracturing of the wellbore to promote increased flow, and other well and wellbore treatments.

A “production plan” can include placement of wells, length of well, depth of well, completion details, enhanced oil production methods, stimulation methods, fracking methods, order of completion, production rate, and the like. Production plans include well stacking, well spacing, completion designs (frac job types, job size, number of stages, stage length, number of clusters per stage, etc.) and strategies (e.g., at what sequence to frac different target zones, how to synchronize/coordinate with nearby wells), production well pressure management, and the like.

An “optimized” production plan is generated using well modeling and predictions to improve the simulated production from a well. Once a well plan is optimized, it may then be implemented at the well, at a well pad with multiple wells, or in an area penetrating one or more reservoirs.

As used herein, to “implement” an optimized plan, means to perform the optimized production plan to drill and/or produce one or more wells in the reservoir, and thereby produce more hydrocarbon than would be produced if the plan was not optimized.

The term “matrix flow” refers to the movement of reservoir fluids through the rock matrix. “Fracture flow” refers to the movement of reservoir fluids through natural or induced fractures or fracture networks in the rock.

As used herein, “landing zone” refers to location within a strata where a lateral (horizontal) well is drilled.

As used herein, “SARA fractions” refers to the four fractions (%) of crude oil that can be separated, including saturates, aromatics, resins and asphaltenes. SARA quantification is typically performed by IP-143 and ASTM D893-69 standards.

As used herein, “water cut” is the ratio of water produced compared to the volume of total liquids produced from a given well.

As used herein, “Gas Oil Ratio” or “GOR” is the ratio of gas volume to oil volume at a given pressure and temperature condition (typically surface conditions) when petroleum is produced from reservoir. It is generally related to the relative quantities of gas and liquid hydrocarbons in the petroleum reservoir, but heavily influenced by reservoir and production well pressure and temperature conditions. It is typically represented as standard cubic feet per stock tank barrel (scf/stb).

The “associated gas” is natural gas that is dissolved in the oil and is produced along with the crude oil. Heavy crude oil has low API gravity and low capacities of dissolved gas as compared to lighter crude oil.

“Steam to Oil Ratio” or “SOR” is a measure used to quantify the efficiency of production of oil from a heavy oil reservoir via injecting steam. It can be defined as the amount of steam injected to produce one unit volume of crude oil. The steam is quantified by barrels of water used to make the steam, however. For example, a steam-oil ratio of 4.5 means that 4.5 barrels of water—converted into steam and injected into the well—were required to extract a single barrel of oil.

“API gravity” measures the relative density of petroleum liquid and water and has no dimensions. To derive the API gravity, the specific gravity (SG) is first measured using either the hydrometer, detailed in ASTM D1298 or with the oscillating U-tube method detailed in ASTM D4052. The official formula used to derive the gravity of petroleum liquids from the SG, as follows: API gravity=141.5/SG−131.5.

“Samples” may include oil, gas, core samples, drilling cuttings, drilling mud, formation fine, produced water, fluids extracted from rock or core, portions thereof, and combinations thereof.

A “core” or “rock core” is a sample of rock, typically in the shape of a cylinder, taken from the side of a drilled oil or gas well. A core is then dissected into multiple core plugs, or small cylindrical samples measuring about 1 inch in diameter and 3 inches long.

“Drilling cuttings” or “cutting samples” are the small irregular rock samples generated during drilling and returned with the drilling mud.

By “obtaining” a sample herein we do not necessarily imply contemporaneous sampling procedures as existing samples can be used where available. However, often contemporaneous sample collection will be needed, except for core or cutting samples, which may already be available.

By generating a reservoir “map” we mean that the reservoir is characterized in the three directional axes, as well as possibly the fourth time axis, but we do not necessarily imply a graphical representation thereof, as data can be maintained and accessed in many forms, including in tables. The map maybe segmented into zones, where the fingerprinting data is very similar.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention. Any claim or claim element introduced with the open transition term “comprising” may also be narrowed to use the phrases “consisting essentially of” or “consisting of.” However, the entirety of claim language is not repeated verbatim in the interest of brevity herein.

The following abbreviations may be used herein:

| Abbreviation | Term |
|---|---|
| AAS | Atomic absorbance spectrophotometer |
| API | American Petroleum Institute, also API gravity, is a |
| | measure of how heavy or light a petroleum liquid is |
| | compared to water: if its API gravity is greater than |
| | 10, it is lighter and floats on water; if less than 10, |
| | it is heavier and sinks. |
| Bbl | Barrel |
| BTU | British thermal unit-definitions vary but in natural |
| | gas pricing a BTU is the amount of heat required to |
| | raise the temperature of 1 avoirdupois pound of pure |
| | water from 58.5 to 59.5° F. (14.7 to 15.3° C.) at a |
| | constant pressure of 14.73 pounds per square inch. |
| BVO | Bulk volume oil |
| CAI | Conodont alteration index |
| CE | Capillary electrophoresis |
| CGR | Condensate gas ratio-CGR gives a measure of the liquid |
| | content to the volume of gas. It is measured in barrels |
| | per millions of standard cubic feet (barrels/mmscf). |
| CHNOS | Carbon, hydrogen, nitrogen, oxygen and sulfur |
| COP | Cumulative oil production |
| CPI | Carbon preference index |
| CSIA | Compound specific isotope composition |
| CUM | Cumulative |
| E&P | Exploration and Production |
| FAMM | Fluorescence alteration of multiple macerals |
| FTICR | Fourier transform ion cyclotron resonance |
| FTIR | Fourier transform infra-red |
| GC | Gas chromatography |
| GC-IR-MS | Gas chromatography isotope ratio mass spectrometry |
| GCxGC | 2D gas chromatography |
| GOR | Gas to oil ratio-When petroleum is produced to surface |
| | temperature and pressure it usually forms two phases: |
| | gas and oil (liquid). The gas/oil ratio (GOR) is the |
| | ratio of the volume of gas to the volume of oil at |
| | specified conditions (typically Temperature = 273.15 K, |
| | Pressure = 1 bar) |
| GR | Gamma ray |
| GUI | Graphical user interphase |
| HPLC | High pressure liquid chromatography |
| HPS | High pressure separator |
| HRGC | High resolution GC |
| IC | Ion chromatography |
| ICP-MS | Inductively coupled plasma MS |
| IRLS | Isotope ratio laser spectroscopy |
| IRMS | Isotope ratio mass spectroscopy |
| KIE | Kinetics isotope effect |
| MAE | Mean absolute error |
| MPLC | Medium pressure liquid chromatography |
| MS | Mass spectrometry |
| NMR | Nuclear magnetic resonance |
| OEP | Odd-even predominance |
| PLT | Production logging test |
| PV | Pressure volume |
| PVT | Pressure volume temperature |
| SARA | Saturates, aromatics, resins, asphaltenes |
| SCF | Standard cubic foot |
| SG | Specific gravity |
| SOR | Steam to oil ratio |
| TAI | Thermal alteration index |
| TLC | Thin layer chromatography |
| TLG | Time lapse geochemistry-geochemical fingerprints |
| | taken from a plurality of samples collected over time |
| TOC | Total organic carbon |
| TOFMS | Time of flight mass spectrometry |
| XRF | X-ray Fluorescence |

### BRIEF DESCRIPTION OF THE DRAWINGS

FIG. 1. Sequential stepwise reaction network devised to model the bulk and isotope composition evolution of petroleum.

FIG. 2. Kinetics modeling of bulk composition evolution detailed to individual alkane.

FIG. 3. Kinetics modeling results of gas oil ratio (GOR) changes over maturity.

FIG. 4. Kinetics modeling results of gas wetness changes over maturity.

FIG. 5. Pseudo gas chromatograms of an oil at different maturities as an illustration of the bulk composition evolution with kinetic modeling.

FIG. 6. Kinetic modeling results of compound specific isotope composition changes over maturity.

FIG. 7. Chung's plot.

FIG. 8. Kinetic modeling of isotopologue ratio changes over maturity.

FIGS. 9A and 9B. Workflow.

### DETAILED DESCRIPTION OF THE DISCLOSURE

The determination of the most favorable petroleum exploration targets depends on the geochemical knowledge of source rocks and on the knowledge of generation, migration and accumulation processes combined with the geophysical and geological features of the sedimentary basin under evaluation. Most crude oil and gas are derived from sedimentary organic matter with kerogen as the major intermediate. During the process of thermal maturation, kerogen reacts to form lower-molecular-weight products, including bitumen, oil, and gas.

At present, the methods to determine the thermal maturity of oil source rocks—a key consideration in oil and gas exploration—are mainly classified into two types. One type of method studies the parameters of the evolutionary characteristics of kerogens, such as vitrinite reflectance (% R_{O}), rock pyrolysis, fluorescence alteration of multiple macerals (FAMM), the degree of spore-pollen carbonization, thermal alteration index (TAI), conodont alteration index (CAI), kerogen elemental composition, and the like. These methods, despite being of relatively high accuracy, have drawbacks such as requirement of large sample amount, complex testing procedures and extended duration.

Another approach studies the parameters of the evolution characteristics of petroleum fluid, such as carbon preference index (CPI), odd-even predominance (OEP), biomarker compound parameters, such as sterane and terpene isomerization ratio, and the like. These are mainly used for qualitative evaluation, since precision is lacking in these methods.

Herein we use geochemical fingerprinting of both bulk compositions and isotope compositions of a given petroleum fluid to assess fluid maturity, and to elucidate charge history of a given reservoir. Maturity assessment based on biomarkers is typically only applicable to lower maturity fluids, because biomarkers are largely absent in the high maturity fluids. Some of the light end hydrocarbons in a reservoir may originate from either secondary cracking of the in-situ oil or from later charge of high maturity fluids, or a mixing of both mechanisms. Thus, conventional maturity assessment based on the isotope compositions of methane, ethane and propane is inadequate to fully define the fluid maturity and unravel the charge history.

We present novel kinetic models to describe/model the petroleum fluid evolution with respect to its bulk compositions and the isotope compositions. The bulk compositions are detailed to individual n-alkanes, while the isotope compositions are detailed to individual isotopomers in each isotopologue of a given n-alkane. This provides a novel systematic solution to assess fluid maturity and to elucidate the charge history of a reservoir, based on the distribution of n-alkanes and detailed isotope composition of each n-alkanes. Applying these models can better predict and understand production performance of a reservoir, like production GOR and produced gas wetness, thus assist in play evaluation and development optimization.

The novel kinetics method which models petroleum evolution bulk composition detailed to each component alkane, and isotope composition detailed to each isotopomer within an isotopologue of a given alkane is described next.

### Modeling 4

The generation and subsequent compositional change of petroleum can be modelled kinetically via a series of sequential cracking reactions (cleavage of C—C bond) over a given thermal history. In primary cracking, alkane molecules break off from kerogen. In secondary cracking, alkane molecules undergo progressive chain length reduction by breaking into smaller alkanes.

There are two carbon isotopes occurring in petroleum—¹²C with a natural abundance around 99% and ¹³C with a natural abundance around 1%. ¹³C substitution leads to a very subtle change in the C—C bond strength, and thus affects the bond cleavage reaction (referred as kinetic isotope effect or “KIE”), and eventually this will affect the ¹³C content in petroleum molecules. The method described herein holistically describes and models the composition evolution via a series of coupled chemical reactions, at a level down to individual C—C bonds of a given alkane molecule.

The modeling procedure is described below. All steps detailed below were implemented in our proof-of-concept work with C++ (and PYTHON for data visualization), but any suitable code may be used, such as FORTRAN, COBOL, BASIC, PASCAL, JAVA, etc. A large list of programming languages is available at en.wikipedia.org/wiki/List_of_programming_languages (incorporated by reference in its entirety for all purposes).

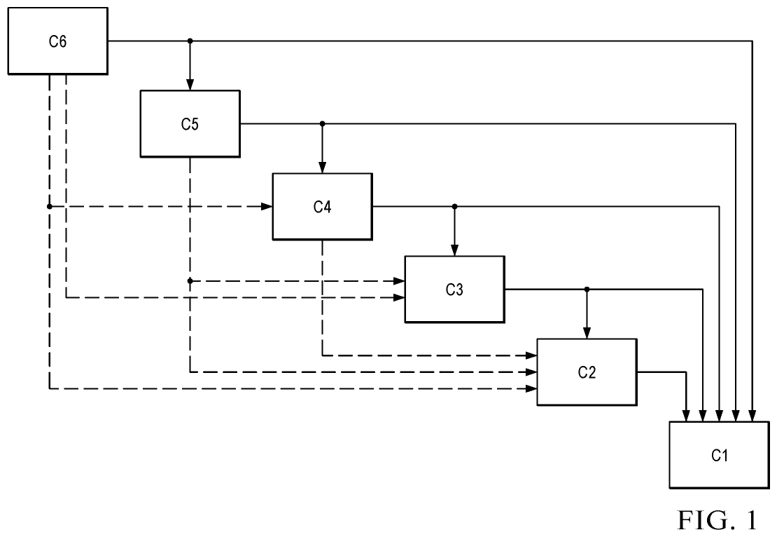

1) Devise a reaction network that describes the evolution of a hydrocarbon composition, as illustrated in FIG. 1. Larger alkanes can crack to smaller alkanes via breaking any C—C bond, but only a single bond may break in each step of the reaction. For example, in a given step of reaction, one hexane (C6) molecule can break into one pentane (C5) molecule and one methane (C1) molecule, or into one butane (C4) molecule and one ethane (C2) molecule, or two propane (C3) molecules.

2) Permutate all possible isotopic isomers for each alkane reactant. Calculate the statistical abundance of each isotopic isomer based on bulk carbon isotope composition of the alkane. For example, let L (for light carbon) stand for ¹²C and H (for heavy carbon) stand for ¹³C, from left to right, all isotopic isomers of propane (C3) are listed as: LLL, HLL, LHL, LLH, HHL, HLH, LHH, HHH. Based on the bulk carbon isotope composition (δ¹³C) of propane, calculate the statistical abundance of each isotopic isomer.

The actual calculation of the statistical abundance is easy. For example, if we have unlimited number of marbles where 1% of them are red and the rest are blue, and we randomly draw one marble, 99% chance it will be blue, 1% red. If we randomly draw two marbles, the chance for BB, BR, RB, RR will be 98.01%, 0.99%, 0.99%, 0.01%, respectively. Nevertheless, strictly speaking, it is supposed to initialize the relative abundance of all isotopic isomers with the thermodynamic equilibrium concentrations at time zero before reaction starts. The thermodynamic equilibrium itself is another branch of established research, and the system may never reach thermodynamic equilibrium, as known by experienced isotope geochemists. Here, we use statistical abundance as an approximation to thermodynamics equilibrium abundance, which might be off a bit but not by much, as eventually it approximates statistical probability. But this is a vital differentiating step from the prior art-we begin with a distribution of abundances for different isotopic isomers, compared to assuming random substitution of ¹³C within a hydrocarbon molecule (as described in the prior art).

3) For each isotopic isomer (species), list out all possible cracking products. For example, species HLL may produce H and LL, or HL and L upon cracking. Based on this information, build out a stoichiometry matrix for the entire reaction network.

4) Describe each step of each reaction (cleavage of each C—C bond in an alkane molecule) with the Arrhenius equation (k=Ae^{(-Ea/RT)}) by specifying A (frequency factor) and Eₐ (activation energy) according to the size of the alkane, the position of the C—C bond, and the identity of carbon atom at either side of the bond (¹²C or ¹³C) and the identity of the carbon atom immediately next to this bond (if any). If desired the alternate version of the Arrhenius equation may be used here.

5) Build out the needed differential equations governing the concentration changes of all species involved, based on the stoichiometry matrix built out in step 3 and reaction rate constant (k) built out in step 4. For example:

$$\frac{d {C}_{p}}{d t} = {S}_{r , p} {k}_{r , p} {C}_{r}$$

wherein S_{r,p} is a stoichiometry coefficient for the reaction from reactant r to product p and k_{r,p} is a reaction rate constant for the reaction from reactant r to product p. Cᵣ is the concentration of reactant r, Cₚ is the concentration of product p and t is time.

6) Solve the coupled differential equations over time for concentration of each species, at a specified thermal history setting, like heating rate and initial temperature, using the initial concentration and isotopic isomer relative abundances calculated in step 3. Here we are solving differential equations over time with time dependent term k (as reaction temperature is time dependent). Reaction temperature is the temperature of the target rock interval over geological time (thermal history).

7) Calculate the thermal maturity indicator, such as vitrinite reflectance (% R_{O}), associated with the thermal history specified in step 6. Temperature, of course, is explicitly calculated. We could use other maturity indicators as well, but % R_{O} is the most useful and meaningful at this stage. There are well established vitrinite reflectance (% R_{O}) models to calculate maturity over a given thermal history since % R_{O} is the conventional indicator of thermal maturity of petroleum source rock and petroleum fluids. When we have a thermal history specified, we can calculate the corresponding thermal maturity indicator.

8) Sum (add) the concentration of each species by carbon number to obtain bulk composition (at an individual alkane level) evolution over time. For each carbon number, sum all the species to obtain the bulk compositions of the petroleum fluid, expressed in terms of concentrations of C1, C2, C3 . . . . C40 (or to larger carbon numbers as needed).

9) For each alkane (by carbon number), calculate the compound specific isotope composition (CSIA) based on concentration of each isotopic isomer and its number of ¹³C substitutions. For example:

$${R}_{n} = \frac{{\sum }_{i = 1}^{k} {s}_{i} {C}_{i}}{{\sum }_{i = 1}^{k} ( n - {s}_{i} ) {C}_{i}}$$

wherein Rₙ stands for ¹³C/¹²C ratio for alkane with n carbon number, which has in total k isotopic isomers. sᵢ stands for number of ¹³C substitution within isomer i, which has a concentration of Cᵢ.

From Rₙ, the δ value for alkane with n carbon number can be calculated with equation below, in which R_{std} stands for the ¹³C/¹²C ratio of standard material.

$$δ = 1000 ( \frac{{R}_{n}}{\text{?}} - 1 )$$

$$\text{?} \text{indicates text missing or illegible when filed}$$

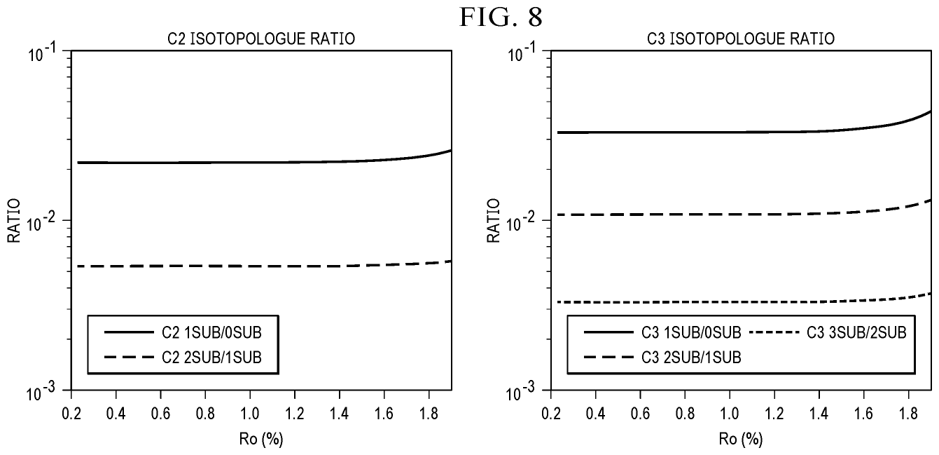

10) Select one or more isotopologues and/or isotopomers for more specific analysis and applications, e.g., changes in relative abundances (ratios) of isotopologues over maturity (FIG. 8), and site-specific isotope analysis within an isotopologue.

For example, there are three (four in the mathematic treatment) isotopic isomers of ethane, we sum them together to calculate the overall carbon isotope signature (δ¹³C) for ethane. In addition, we have the concentrations of each individual isotopic isomer of ethane, which allow us to pursue higher (or deeper) levels of investigation. For example, two petroleum samples may have very similar δ¹³C value of ethane, but the individual ethane isotopic isomer concentrations may be different for these two samples. For propane, with one ¹³C substitution, there are two isomers (three in the mathematic treatment), corresponding to terminal and center substitution. For a given precursor composition and thermal history, the kinetics model can calculate the abundance of these isomers at different maturity stages. Site specific isotope analysis can determine the abundance of these two (terminal vs. center) isomers for a gas sample. The measurement can shed light on the modeling result (calibration), and the model can be used to make predictions or rationalize observations in petroleum E&P operations.

11) Model the thermal maturity (% R_{O}) with established vitrinite reflectance kinetics models (e.g., EasyRo) or custom vitrinite reflectance kinetics model specific for the source rock and the generated petroleum fluid, for the given thermal history under investigation.

12) Compare/calibrate the model with measured petroleum bulk composition and isotope composition data, and make predictions based on kinetics modeling.

### Applications

There are many applications of this novel kinetics method, including applications for petroleum E&P operations. Applications include but are not limited to:

1) Hydrocarbon source and charge history analysis, maturity assessment, development and production optimization, and production surveillance, especially for well interference from other wells.

2) Petroleum properties (GOR, API gravity) delineation or prediction based on complete composition of alkanes at various maturity stages from kinetics modeling. Once having the complete composition, calculation of GOR and oil API gravity is straightforward. GOR is approximated with the ratio of C1-C5 over C6+. API is largely based on the average molecular weight of C6+ fraction.

3) Export the complete composition of alkanes at various maturity stages from the kinetics modeling for PVT property modeling, using e.g., PVTSim software which takes petroleum compositions, traditionally from GC analysis, in this case from kinetic modeling, for petroleum PVT properties predictions.

4) For a given play (typically laterally continuous unconventional plays like Eagle Ford, Montney, etc.), based on the compositions (both bulk and isotope) of a less mature oil (e.g., oil in up dip shallower portion of the play), simulate the composition of the less mature oil upon secondary cracking to different stages of maturity, and compare the simulated compositions to oil in down dip deeper portion of the play. If the simulated compositions match down dip oil, it suggests the down dip more petroleum resulted from the secondary cracking of the less mature oil initially charged into the reservoir. If the simulated compositions do not match down dip oil, it suggests the down dip reservoir might have received additional petroleum charges from another source.

Additional applications may include those in Table 1.

TABLE 1
Additional applications for the technology described herein

Fluid correlation, charge history analysis, production allocation
and production forensics with bulk and isotope compositions of
extended list of alkanes.
New thermal maturity measurement/assessment based on detailed
isotope compositions of an extended list of petroleum compounds.
Burial/thermal/maturation history reconstruction and kitchen evaluation.
Interpretation of paleothermal indicators and thermal regimes.
Assessment of source rock effectiveness.
Assessment of shale potential and identification of sweet spots.
Volumetric estimation of oil and gas generated, expelled, migrated,
accumulated.
Understanding of oil families and their corresponding sources.
Ranking of prospects (size, petroleum fluid characteristics, uncertainty).
Understanding expulsion efficiencies.
Pore pressure history, prediction of present-day overpressures
before drilling.
Reconstruction of oil and gas migration pathways and migration
efficiencies.
Role of faults in oil and gas migration.
Understanding of origin of gas: biogenic, thermogenic, or
non-hydrocarbon gas (CO₂).
Understanding seal efficiency and oil/gas column heights.
Postmortem interpretations.
Delineation and development of exploratory areas.
Understanding of oil heterogeneities at field scale.

### EXPERIMENTAL

Samples—Core, cutting and mud gas samples were collected during drilling. Produced fluids (oil, gas, water) samples were collected over time once the well comes online. In addition, rock samples may be collected from outcrop and fluids samples may be collected from seeps.

Rock samples can be solvent extracted either with dichloromethane (CH₂Cl₂) or carbon disulfide (CS₂) for better preservation of the light end hydrocarbons. Light end hydrocarbons from rock samples can also be thermally extracted via low temperature pyrolysis. Gas samples are analyzed for bulk compositions and compound specific isotope compositions. Whole oil GC and compound specific isotope analyses are performed on produced oil samples and rock extracts. We try to analyze the full carbon number range in the oil and extract. For CS₂ extract, it can go up to 41 carbons (where our typical GC analysis ends).

Analysis of Samples—the samples are then analyzed by one or more methods to provide accurate fingerprints of the contents. We have exemplified the methods herein using isotope ratio mass spectrometry (IRMS), but any methods or combinations of methods can be used, including Gas Chromatography (GC), Two-dimensional Gas Chromatography (GCxGC), Mass Spectrometry (MS), Tandem mass spectrometry (MS/MS), GC-IRMS, GC-MS, GC-MS/MS, GCxGC-MS, GCxGC-IRMS, thin layer chromatography (TLC), including 2D TLC, capillary electrophoresis (CE), High Pressure Liquid Chromatography (HPLC), Fourier Transform Infra-Red (FTIR) Spectrophotometer, X-ray Fluorescence (XRF), Atomic Absorbance Spectrophotometer (AA or AAS), Inductively Coupled Plasma Mass Spectrometry (ICP-MS), Ion Chromatography (IC), Nuclear Magnetic Resonance (NMR), Fourier Transform Ion Cyclotron Resonance mass spectrometry (FTICR-MS), and the like. Additional analysis can include gas compounds, isotopes, bulk oil parameters (API gravity, SARA, CHNOS, elemental), and the like.

Bulk oil and gas property analyses can also be applied, including but not limited to PVT properties (phase behavior properties), density, specific gravity, API gravity, viscosity, surface tension, interfacial tension, carbon residue, aniline point, specific heat, heat content, enthalpy, heat of combustion, electrical conductivity, dielectric constant, dielectric strength, refractive index, optical properties, fractional composition (saturates, aromatics, resin and asphaltenes, aka SARA composition), simulated distillation, adsorption chromatography, gel permeation chromatography, ion-exchange chromatography, high-performance liquid chromatography, supercritical fluid chromatography, thin layer chromatography, structural group analysis, molecular weight, mixed aniline point, correlative methods, evaporation rate, boiling point, pour point, cloud point, flash point, Kauri-butanol value, odor, color, volatility, storage stability, thermal stability, and the like.

In some cases, it may be necessary to separate the samples into two or more fractions before submitting sub-fractions to fingerprinting analysis, because oil components can range from C1 to >C40 in some samples, and because there are instances where certain components can interfere with a particular analysis. However, fingerprinting and chemical analyses are well known in oil and gas exploration and production, and the person of ordinary skill knows how to apply a correct methodology to a given sample type.

Data Consistency: Fingerprint data can have considerable variability depending on the machines used, operator technique, method of sample collection, storage conditions, age of samples, and the like. Therefore, steps should be taken to ensure internal consistency such that the data is more reliable, including e.g., comparing multiple phases (gases, low and high molecular weight oil fractions), establishing a standardized protocol for sample collection with single vendor, immediate sample analysis on arrival to minimize aging errors or the use of suitable verified storage conditions, and of course, running external standards and performing fluid analyses in duplicate, triplicate, or better.

In addition, oil and gas can be analyzed at different laboratories and on different instruments for the same wells. By comparing the same samples analyzed in different labs and/or different machines, a margin of error can be established, and if needed stable datapoints can be selected for use in the subsequent analysis and the more variable data elements omitted.

### Results

To illustrate the modeling of bulk composition evolution of petroleum fluids, let's assume a simple petroleum generation system beginning with a long aliphatic chain (C41), which functions as the surrogate for the labile fraction of kerogen involved in petroleum generation. The primary cracking is the generation of alkanes of different chain lengths from kerogen, from as big as the entire aliphatic chain defined at beginning (breaking the bond connecting the entire labile chain to inert portion of the kerogen structure), to as small as methane (breaking the bond at the other terminal of the long aliphatic chain). All primary cracking reaction proceeds at rate according to the location of the C—C bond under cracking within the aliphatic chain. Once generated, each individual alkane (except methane) undergoes cracking following the rules listed in the method description section.

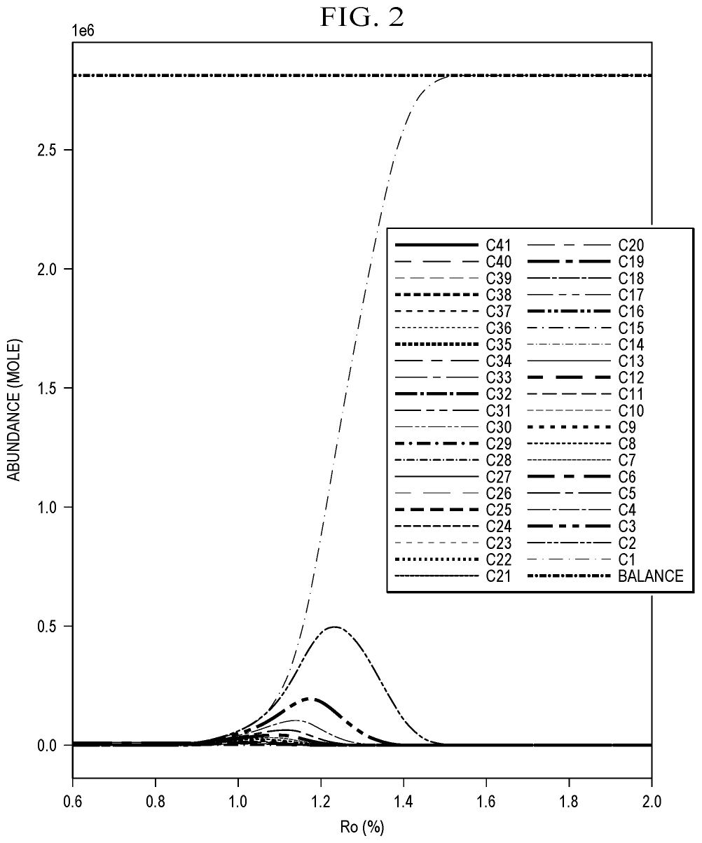

FIG. 2 shows the modeling results of bulk composition evolutions over maturity (vitrinite reflectance) for a petroleum fluid generated from an aliphatic chain of 41 carbons, modeled with a vitrinite reflectance kinetics model. With the ability to handle individual C—C bond based on its position in any carbon chain, this kinetic method allows modeling of detailed composition evolution down to individual alkane of different chain lengths. Other current kinetics modeling methods are only capable of modeling the bulk composition evolution by dividing the petroleum into a few fractions, like C16+, C15-C6, C5-C2, and C1. This method represents a significant advent in terms of compositional complexities encompassed. With such detailed compositional modeling, key bulk fluids properties like GOR and gas wetness can be easily derived.

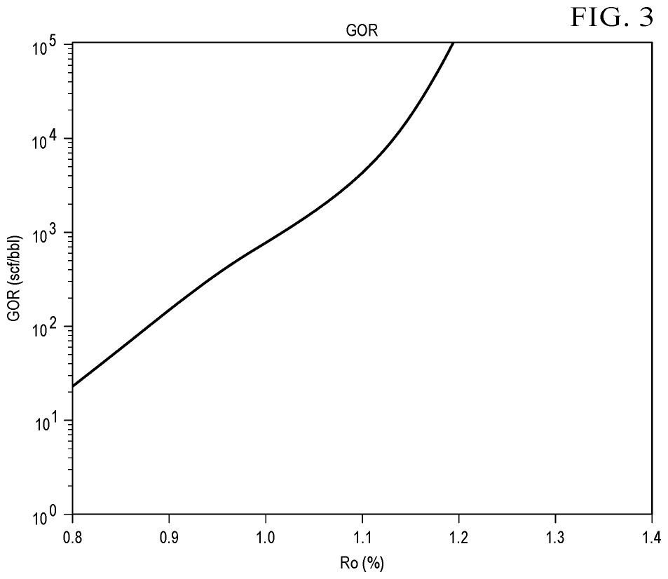

FIG. 3 shows the cumulative GOR changes corresponding to the bulk composition evolution modeled in FIG. 2 done by summing light end (gas fraction) over oil fraction, then with some unit conversions.

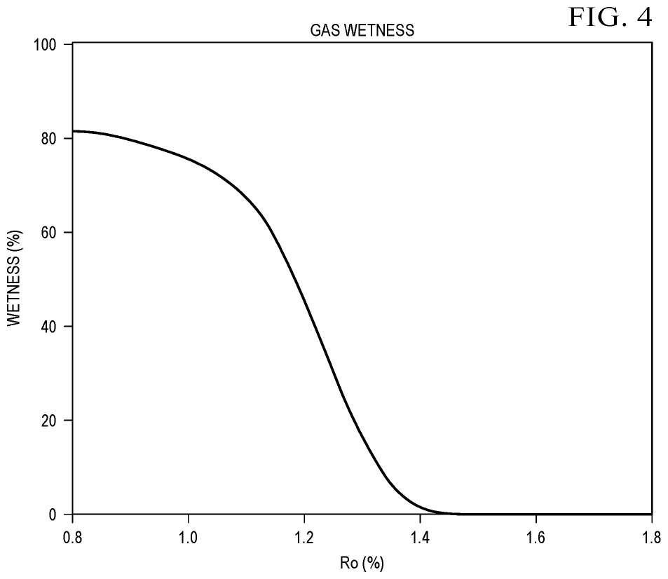

FIG. 4 shows the gas wetness changes associated with the bulk composition evolution modeled in FIG. 2. Please note these figures were generated for illustration purposes. The modeling results can vary significantly depending on the settings of kinetics parameters. There are a lot of kinetic parameters, as each reaction has its own rate constant k; k is defined by Arrhenius equation with two key parameters, A and Ea. Optimization of A and Ea is another task. Also, practically speaking, this model is currently doable for a relatively small system. However, as computation technology continues to advance, larger systems will be readily modeled.

Another particularly useful application of this kinetics method is to model the composition changes of a migrated oil upon secondary cracking. By setting initial reactants to be a suite of alkanes, whose relative abundance can be determined by GC analysis of a low maturity oil, this kinetics method can model the subsequent bulk composition evolutions by subjecting the initial suite of alkanes to secondary cracking to various maturity stages.

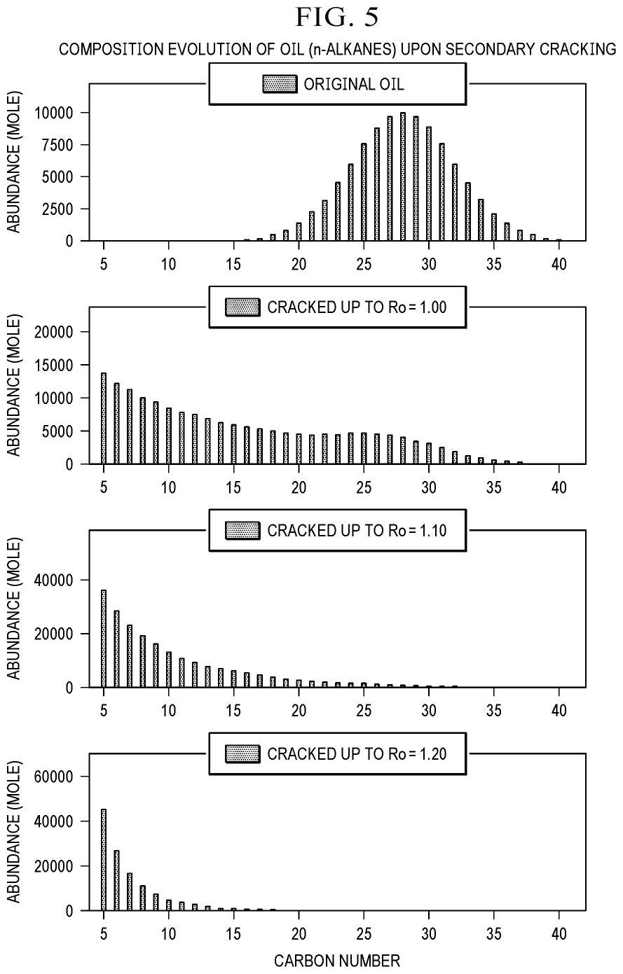

As an illustration, FIG. 5 shows the pseudo-GC (or approximated) chromatograms of the initial oil prior to secondary cracking (top), and the corresponding changes upon secondary cracking to different extents (using % R_{O} as an indicator of different levels of secondary cracking).

Again, the result shown here is to illustrate the functionality of this kinetic method, the actual modeling result can vary significantly depending on actual settings of kinetics parameters. However, together with the bulk composition evolution modeling described in the preceding paragraph (primary cracking plus secondary cracking in source kitchen), this kinetic modeling method supplies a powerful solution for charge history analysis by running different scenarios.

For example, a single charge case vs. an initial oil charge plus later gas charge case would have different bulk composition and isotope compositions. Sometimes, based on oil bulk composition profile, our understanding of the source rock facies (oil prone or gas prone source rocks), maturity, GOR and reservoir PT conditions, we may assess the charge history, but often run into situation that analysis/identification based on bulk composition profile is inadequate. Under such circumstances, isotope composition clarifies the situation, and it is more definitive than bulk oil/gas composition alone. The isotope composition modeling described next can further enhance the charge history analysis. We may have a source rock bed along a dip, the down-dip section is mature, the up-dip section is immature. We call the mature portion a “source kitchen” which are pods of source rocks mature enough to generate hydrocarbons. Oil to gas cracking may happen in reservoir as well.

To systematically model the kinetics isotope fractionations accompanying the bulk composition evolutions of a petroleum mixture, this method takes all possible ¹³C substitution scenarios for a given alkane into account. It is fundamentally different to, and more computationally challenging than, other kinetic isotope fractionation modeling methods that assume only one ¹³C substitution per molecule.

There are mainly two challenges associated with the method presented herein. The first one is the determination of initial relative abundances of all isotopic isomers for a given alkane. At this stage—the very beginning of this effort—we assume the relative abundances of all isotopologues for a given alkane follow statistic probabilities. Within each isotopologue, each isotopomer has equal abundance. Using nC12 with a bulk carbon isotope composition (δ¹³C) of −30% % as an example, Table 2 summarizes the statistical abundances of each isotopologue (based on number of ¹³C substitutions), number of isotopomers within each isotopologue, and the cumulative δ¹³C values (take each isotopologue into account, with ¹³C substitutions from low to high).

TABLE 2
Number and abundance of carbon isotopologues of nC12 with bulk δ¹³C of −30‰
Statistical isotopic isomer abundances for n-alkane with 12 carbon atoms and
bulk carbon isotope composition (δ¹³C) f −30.00 per mille

| 13C | | Num. of | | |
|---|---|---|---|---|
| Subst. | Rel. Abundance | Isomers | Cum. 13C Pool | Cum. Delta 13C |
| 0 | 8.78013530E−01 | 1 | 0.00000000E+00 | −1000.00000000 |
| 1 | 1.14845055E−01 | 12 | 8.87583951E−01 | −133.85195135 |
| 2 | 6.88501409E−03 | 66 | 9.94006087E−01 | −35.62714046 |
| 3 | 2.50157440E−04 | 220 | 9.99806138E−01 | −30.18397401 |
| 4 | 6.13515849E−06 | 495 | 9.99995802E−01 | −30.00401065 |
| 5 | 1.0699798E−07 | 792 | 9.99999936E−01 | −30.00006120 |
| 6 | 1.36066824E−09 | 924 | 9.99999999E−01 | −30.00000067 |
| 7 | 1.27126270E−11 | 792 | 1.00000000E+00 | −30.00000001 |
| 8 | 8.66054386E−14 | 495 | 1.00000000E+00 | −30.00000000 |
| 9 | 4.19558469E−16 | 220 | 1.00000000E+00 | −30.00000000 |
| 10 | 1.37196677E−21 | 66 | 1.00000000E+00 | −30.00000000 |
| 11 | 2.71900964E−21 | 12 | 1.00000000E+00 | −30.00000000 |
| 12 | 2.46978612E−24 | 1 | 1.00000000E+00 | −30.00000000 |

This assessment indicates that assuming only one ¹³C substitution per molecule is greatly inadequate to capture the characteristics of carbon isotope fractionation in petroleum. Though the real abundance distribution for a given alkane may not be completely random, and thus will to some degree deviate from the statistical abundances calculated herein, the method present herein is still far more realistic and technically sound than assuming one or two ¹³C substitution per molecule, especially as the molecule gets larger.

However, as alkane molecule gets larger (carbon chain length increases), the number of isotopic isomers increases rapidly. This entails constructing and solving an exceedingly large number of coupled differential equations, which could be a formidable numerical challenge as the modeled system gets big (in terms of total number of species accounted).

Herein, while we did model up to C41, we did so with reduced numbers of ¹³C substitutions. With a full range of substitutions, the number of differential equations is quite big-over a billion of coupled differential equations. In reality, we typically work with light oil cracking to gas, and the advanced isotope analysis methods are typically only applicable to small hydrocarbon molecules. With improving computational ability, we expect that we will be able to continually expand/enhance the modeling capability.

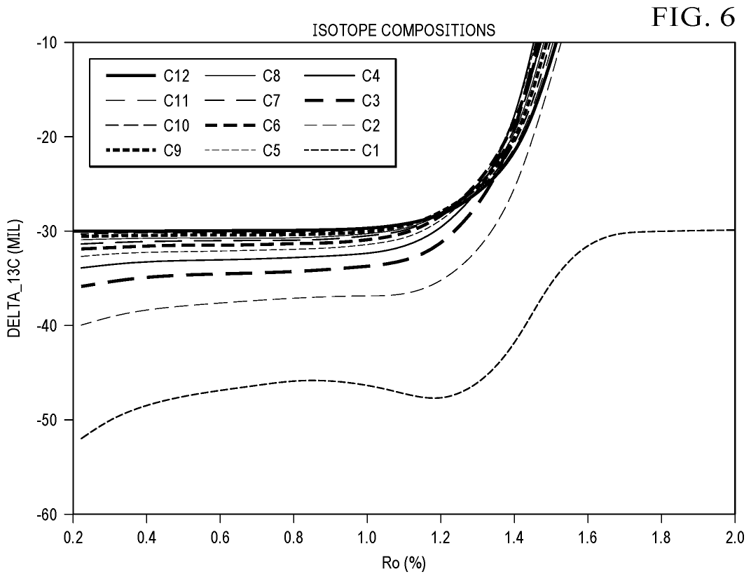

FIG. 6 shows the kinetic modeling results for the compound-specific (in terms of chain length of an alkane, from C1 to C12) isotope composition evolutions upon cracking of nC12 with initial isotope compositions described in Table 2. Here, compound-specific isotope composition is calculated by summing all isotopomers' contribution to the overall ¹³C abundance for a given alkane at a given maturity stage, with the underlying kinetics isotope fractionation effect being exhaustively implemented in each step of cracking reaction for all isotopomers within each isotopologue of the given alkane.

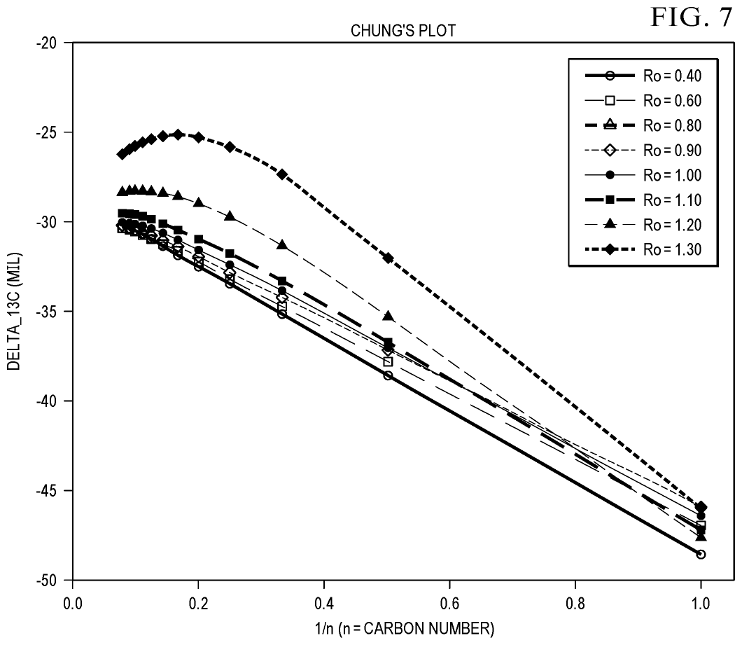

FIG. 7 is another way to present the modeling results shown in FIG. 6. Known as Chung's plot, the bulk isotope composition of an alkane is plotted against the reciprocal of its carbon number (1/n). At a lower maturity stage, the plot approximates a straight line, as predicted by Chung, whose method assumes up to one random ¹³C substitution in an alkane molecule. However, at higher maturity stage, the plot deviates from a straight line, which is more consistent with recent compound-specific carbon isotope analysis results of produced high maturity fluids. From this we have concluded that our approach is better than the prior art random single ¹³C substitution approach that dramatically simplifies the system and deviates too far from reality.

FIG. 8 illustrates a unique capability of the kinetics method presented herein, modeling the relative abundance evolution of isotopologues for a given alkane, which better illustrates the underlying kinetic isotope fractionation effects than the bulk isotope composition changes of a given alkane. The left side and right side of FIG. 8 plots the changes of isotopologue ratios for ethane and propane over maturity, respectively. With latest advent in gas chromatography isotope ration mass spectrometry (GC-IRMS) and nuclear magnetic resonance (NMR) spectroscopy, measurement of isotopologue abundances of small alkanes like ethane and propane has become attainable in research labs.

While modeling the abundances of isotopomers is readily attainable with the kinetics method presented here, measurement of individual carbon isotopomer abundance is currently not attainable in labs (except for methane and ethane and propane), due to difficulty in separating isotopomers within an isotopologue and other challenges, like instrument detection sensitivity limit. Fortunately, the overall abundances of species with high ¹³C substitutions are statistically very low (as shown in Table 2), excluding some of them will not introduce significant error with respect to the bulk carbon isotope signature of a given long chain alkane and the isotope compositions of its offspring species upon secondary cracking.

### Workflow

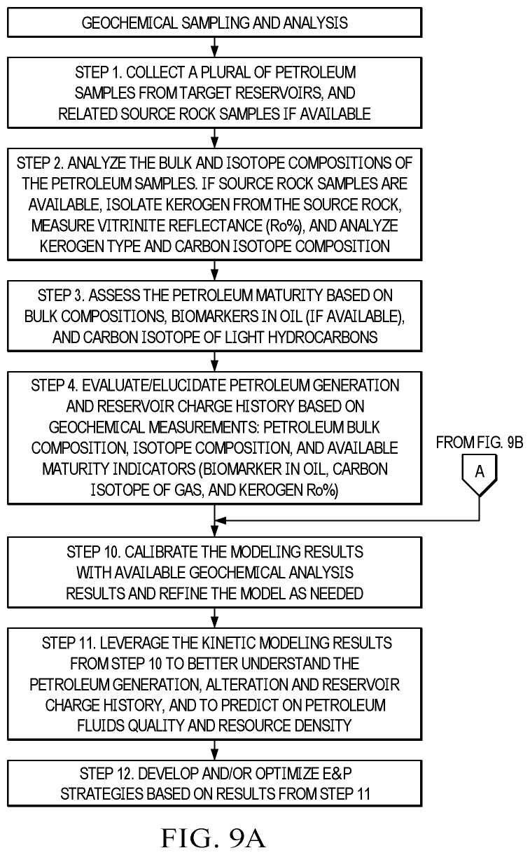

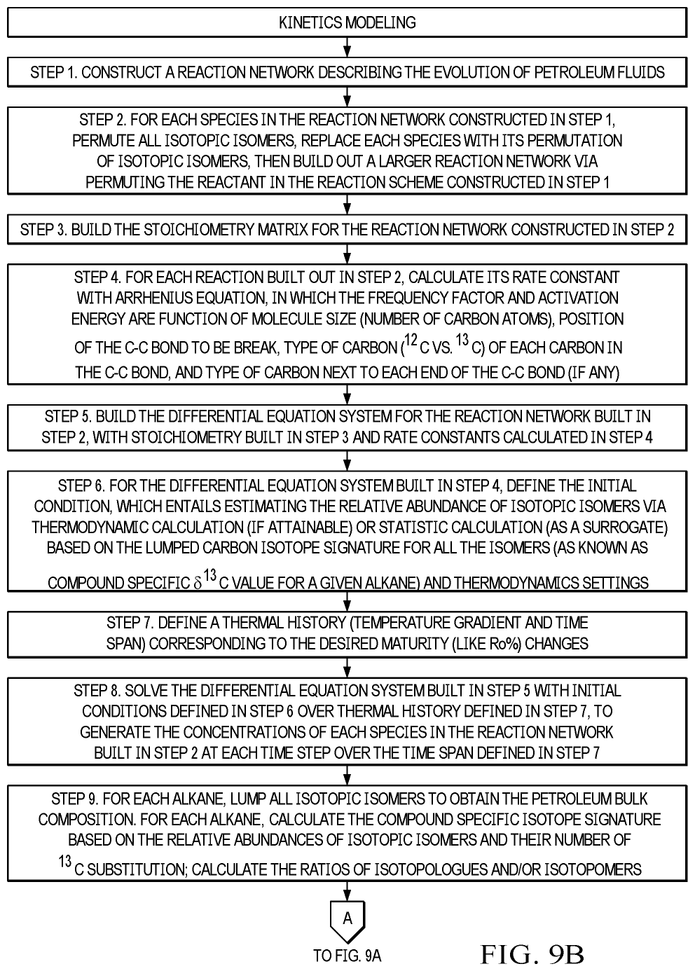

FIG. 9 illustrates the workflow herein. In the geochemical sampling and analysis (FIG. 9A), we show four steps, but of course existing samples and data can be used where available, and these steps may be omittable in whole or part:

Step 1. Collect a plural of petroleum samples (liquid and/or gas) from target reservoirs, and related source rock samples if available, and assign time and location identifiers to each.

Step 2. Analyze the bulk and isotope compositions of the petroleum samples. If source rock samples are available, isolate kerogen from the source rock, measure vitrinite reflectance (% R_{O}), and analyze kerogen type and carbon isotope composition.

Step 3. Assess the petroleum maturity based on bulk compositions, biomarkers in oil (if available), and carbon isotope of light hydrocarbons.

Step 4. Evaluate/elucidate petroleum generation and reservoir charge history based on geochemical measurements: petroleum bulk composition, isotope composition, and available maturity indicators (biomarker in oil, carbon isotope of gas, and kerogen % R_{O}).

In the kinetics modeling (FIG. 9B), there are roughly 12 steps:

Step 1. Construct a reaction network describing the evolution of petroleum fluids.

Step 2. For each species in the reaction network constructed in step 1, permute all isotopic isomers, replace each species with its permutation of isotopic isomers, then build out a multitude of reaction networks via permuting the reactant within the reaction scheme constructed in step 1.

Step 3. Build the stoichiometry matrix for the reaction network constructed in step 2.

Step 4. For each reaction built out in step 2, calculate its rate constant with Arrhenius equation, in which the frequency factor and activation energy are function of molecule size (number of carbon atoms), position of the C—C bond to be break, type of carbon (¹²C vs. ¹³C) of each carbon in the C—C bond, and type of carbon next to each end of the C—C bond (if any).

Step 5. Build the differential equation system for the reaction networks built in step 2, with stoichiometry built in step 3 and rate constants calculated in step 4.

Step 6. For the differential equation system built in step 4, define the initial condition, which entails estimating the relative abundance of isotopic isomers via thermodynamic calculation (if attainable) or statistic calculation (as a surrogate) based on the lumped carbon isotope signature for all the isomers (as known as compound specific δ¹³C value for a given alkane) and thermodynamics settings.

Step 7. Define a thermal history (temperature gradient and time span) corresponding to the desired maturity (like % R_{O}) changes.

Step 8. Solve the differential equation system built in step 5 with initial conditions defined in step 6 over thermal history defined in step 7, to generate the concentrations of each species in the reaction networks built in step 2 at each time step over the time span defined in step 7.

Step 9. For each alkane, lump (sum) all isotopic isomers to obtain the petroleum bulk composition. For each alkane, calculate the compound specific isotope signature based on the relative abundances of isotopic isomers and their number of ¹³C substitution; calculate the ratios of isotopologues and/or isotopomers.

Step 10. Calibrate the modeling results with available geochemical analysis results (from FIG. 9A) and refine the model as needed.

Step 11. Leverage the kinetic modeling results from step 10 to better understand the petroleum generation, alteration and reservoir charge history, and to predict petroleum fluids quality and resource density.

Step 12. Develop and/or optimize exploration and production strategies based on results from step 11.

### Hardware

The present disclosure also relates to a computing apparatus for performing the numerical calculations described herein. All the results presented herein, as proof-of-principle, were obtained from a laptop computer with eight logical CPU cores. All aspects of the kinetics methods presented herein were numerically implemented in C++ (GCC), with massive parallel processing to solve large coupled differential equation systems. Post-processing, like generation of plots, was implemented in Python (Anaconda).

The next step is to perform the calculations on high performance computing (HPC) clusters or cloud with massive parallel processing on larger number of processors. This apparatus may be specially constructed for the required purposes of modeling, or it may comprise a general-purpose computer selectively activated or reconfigured by a spreadsheet program and reservoir simulation computer program stored in the computer. Such computer programs may be stored in a computer readable storage medium, preferably non-transitory, such as, but is not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

In one embodiment, the computer system or apparatus may include graphical user interface (GUI) components such as a graphics display and a keyboard, which can include a pointing device (e.g., a mouse, trackball, or the like, not shown) to enable interactive operation. The GUI components may be used both to display data and process data and to allow the user to select among options for implementing aspects of the method or for adding information about reservoir inputs or parameters to the computer programs. The computer system may store the results of the system and methods described above on disk storage or the hard drive, for later use and further interpretation and analysis. Additionally, the computer system may include one or more processors for running said spreadsheet and simulation programs.

Hardware for implementing the inventive methods may preferably include massively parallel and distributed Linux clusters, which utilize both CPU and GPU architectures. Alternatively, the hardware may use a LINUX OS, XML universal interface run with supercomputing facilities provided by Linux Networx, including the next-generation Clusterworx Advanced cluster management system.

Another system is the Microsoft Windows 10 Enterprise or Ultimate Edition (64-bit, SP1) with Dual quad-core or hex-core processor, 256 GB RAM memory with Fast rotational speed hard disk (10,000-15,000 rpm) or solid state drive (1 TB) with NVIDIA Quadro K5000 graphics card and multiple high resolution monitors.

Slower systems could also be used but is not preferred as solving for multiple equations is time intensive.

Reservoir simulation programs can be any known in the art, possibly modified for use herein, or any novel purpose built system. Existing commercial packages include MEERA, ECLIPSE, RESERVOIR GRAIL, 6X, VOXLER, SURFER, the CMG suite, LANDMARK NEXUS, and the like. Open source packages include BOAST-Black Oil Applied Simulation Tool (Boast), MRST—the MATLAB Reservoir Simulation Toolbox and OPM—The Open Porous Media (OPM).

The following references are incorporated by reference in their entireties for all purposes:
US10400596, US20160084080, Method to enhance exploration, Development and production of hydrocarbons using multiply substituted isotopologue geochemistry, Basin modeling and molecular kinetics.
U.S. Pat. No. 10,415,379, US20160222781, Applications of advanced isotope geochemistry of hydrocarbons and inert gases to petroleum production engineering.
Abrams, M. A.; Thomas D. (2020) “Geochemical evaluation of oil and gas samples from the Upper Devonian and Mississippian reservoirs Southern Anadarko Basin Oklahoma and its implication for the Woodford Shale unconventional play.” Mar. Pet. Geol. 112, 104043.
Behar, F.; Vadenbroucke, M.; Tang, Y.; Marquis, F.; Espitalie, J. (1997) “Thermal cracking of kerogen in open and closed systems: Determination of kinetic parameters and stoichiometric coefficients for oil and gas generation.” Org. Geochem. 26, 321-339.
Boreham, C. J.; Chen, J.; Edwards, D. S.; Hong, Z.; Hope, J. M. (2008) “Carbon and hydrogen isotopes of neo-pentane for biodegraded natural gas correlation.” Org. Geochem. 39, 1483-1486.
Bousige, C., Ghimbeu, C. M.; Vix-Guterl, C.; Pomerantz, A. E.; Suleimenova, A.; Vaughan, G.; Garbarino, G.; Feygenson, M.; Wildgruber, C.; Ulm, F-J.; Pellenq, R., J-M.; Coasne, B. (2016) “Realistic molecular model of kerogen's nanostructure.” Nat. Mater. 15, 576-582.
Burnham, A. K.; Braun R. L. (1990) “Development of a detailed model of petroleum formation, destruction, and expulsion from lacustrine and marine source rocks.” Org. Geochem. 16, 27-39.
Burruss, R. C.; Laughrey C. D. (2010) “Carbon and hydrogen isotopic reversals in deep basin gas: Evidence for limits to the stability of hydrocarbons.” Org. Geochem. 41, 1285-1296.
Byrne, D. J., Barry, P. H.; Lawson, M.; Ballentine, C. J. (2018) “Determining gas expulsion vs retention during hydrocarbon generation in the Eagle Ford Shale using noble gases.” Geochim. Cosmochim. Acta 241, 240-254.
Cesar, J., Eiler, J.; Dallas, B.; Chimiak, L., Grice, K. (2019) “Isotope heterogeneity in ethyltoluenes from Australian condensates, and their stable carbon site-specific isotope analysis.” Org. Geochem. 135, 32-37.
Chatterjee, A.; Vlachos D. G. (2007) “An overview of spatial microscopic and accelerated kinetic Monte Carlo methods.” J. Comput. Mater. Des. 14, 253-308.
Chung, H. M.; Gormly, J. R.; Squires, R. M. (1988) “Origin of gaseous hydrocarbons in subsurface environments: Theoretical considerations of carbon isotope distribution.” Chem. Geol. 71, 97-104.
Clog, M.; Lawson, M.; Peterson, B.; Ferreira, A. A.; Santos, N. E. V.; Eiler, J. M.; (2018) “A reconnaissance study of ¹³C-¹³C clumping in ethane from natural gas.” Geochim. Cosmochim. Acta 223, 229-244.
Cramer, B.; Faber, E.; Gerling, P.; Krooss, B. M. (2001) “Reaction kinetics of stable carbon isotopes in natural gas-Insights from dry, open system pyrolysis experiments.” Energy Fuels 15, 517-532.
Cramer, B.; Krooss, B. M.; Littke, R. (1998) “Modelling isotope fractionation during primary cracking of natural gas: a reaction kinetic approach.” Chem. Geol. 149, 235-250.
Cypres, R. (1987) Aromatic hydrocarbons formation during coal pyrolysis. Fuel Process. Technol. 15, 1-15.
Dai, J.; Zou, C.; Liao, S.; Dong, D.; Ni, Y.; Huang, J.; Wu, W.; Gong, D.; Huang, S.; Hu, G. (2014) “Geochemistry of the extremely high thermal maturity Longmaxi shale gas, southern Sichuan Basin.” Org. Geochem. 74, 3-12.
Domine, F.; Bounaceur, R.; Scacchi, G.; Marquaire, P-M.; Dessort, D.; Pradier, B.; Brevart, O. (2002) “Up to what temperature is petroleum stable? New insights from a 5200 free radical reactions model. Org. Geochem.” 33, 1487-1499.
Dong, G.; Xie, H.; Formolo, M.; Lawson, M.; Sessions, A.; Eiler, J (2021) “Clumped isotope effects of thermogenic methane formation: Insights from pyrolysis of hydrocarbons.” Geochim. Cosmochim. Acta 303, 159-183.
Evans, R. J.; Felbeck G. T. (1983) “High temperature simulation of petroleum formation-I. The pyrolysis of Green River Shale.” Org. Geochem. 4, 135-144.
Fichthorn, K. A.; Weinberg, W. H. (1991) “Theoretical foundations of dynamical Monte Carlo simulations.” J. Chem. Phys. 95, 1087.
Freund, H.; Walters, C. C.; Kelemen, S. R.; Siskin, M.; Gorbaty, M. L.; Curry, D. J.; Bence, A. E. (2007) “Predicting oil and gas compositional yields via chemical structure-chemical yield modeling (CS-CYM): Part 1-Concepts and implementation.” Org. Geochem. 38, 288-305.
Galimov, E. M. (2006) “Isotope organic geochemistry.” Org. Geochem. 37, 1200-1262.
Galimov, E. M. (1988) “Sources and mechanisms of formation of gaseous hydrocarbons in sedimentary rocks.” Chem. Geol. 71, 77-95.
Gao, C. W., et al. (2016) Reaction mechanism generator: Automatic construction of chemical kinetic mechanisms. Comput. Phys. Commun. 203, 212-225.
Gao, L.; Allen, J. W.; Green, W. H.; West, R. H. (2016) “Determination of position-specific carbon isotope ratios in propane from hydrocarbon gas mixtures.” Chem. Geol. 435, 1-9.
Gilbert, A.; Lollar, B. S.; Musat, F.; Ueno, Y. (2019) “Intramolecular isotopic evidence for bacterial oxidation of propane in subsurface natural gas reservoirs.” Proc. Natl. Acad. Sci. U.S.A 116, 6653-6658.
Gilbert, A.; Yamada, K.; Suda, K.; Ueno, Y.; Yoshia, N. (2016) “Measurement of position-specific ¹³C isotopic composition of propane at the nanomole level.” Geochim. Cosmochim. Acta 177, 205-216.
Gilbert, A.; Yamada, K.; Yoshida, N. (2013) “Exploration of intramolecular ¹³C isotope distribution in long chain n-alkanes (C11-C31) using isotopic ¹³C NMR.” Org. Geochem. 62, 56-61.
Gillespie, D. T. (1976) “A general method for numerically simulating the stochastic time evolution of coupled chemical reactions.” J. Comput. Phys. 22, 403-434.
Goldman, M. J.; Vandewiele, N.; Ono, S.; Green, W. H. (2019) “Computer-generated isotope model achieves experimental accuracy of filiation for position-specific isotope analysis.” Chem. Geol. 514, 1-9.
Gonzalez, Y.; Nelson, D. D.; Shorter, J. H.; McManus, J. B.; Dyroff, C.; Formolo, M.; Wang, D. T.; Western, C. M.; Ono, S. (2019) “Precise measurements of ¹²CH₂D₂ by tunable infrared laser direct absorption spectroscopy.” Anal. Chem. 91, 14967-14974.
Hayes, J. M. (2001) Fractionation of carbon and hydrogen isotopes in biosynthetic processes. Rev. Mineral. Geochem. 43,225-277.
He, Y.; Bao, H.; Liu, Y. (2019) “Predicting equilibrium intramolecular isotope distribution within a large organic molecule by the cutoff calculation.” Geochim. Cosmochim. Acta.
Helgeson, H. C; Knox, A. M.; Owens, C. E.; Shock, E. L. (1993) “Petroleum, oil field waters, and authigenic mineral assemblages Are they in metastable equilibrium in hydrocarbon reservoirs.” Geochim. Cosmochim. Acta 57, 3295-3339.
Helgeson, H. C.; Richard, L.; Mckenzie, W. F.; Norton, D. L.; Schmitt, A. (2009) “A chemical and thermodynamic model of oil generation in hydrocarbon source rocks.” Geochim. Cosmochim. Acta 73, 594-695.
Julien, M.; Goldman, M. J.; Liu, C.; Horita, J.; Boreham, C. J; Yamada, K.; Green, W. H.; Yoshida, N.; Gilbert, A. (2020) “Intramolecular ¹³C isotope distributions of butane from natural gases.” Chem. Geol. 541, 119571.
Julien, M.; Nun, P.; Hohener. P; Parinet, J.; Robins, R. J.; Remaud, G. S. (2016) “Enhanced forensic discrimination of pollutants by position-specific isotope analysis using isotope ratio monitoring by ¹³C nuclear magnetic resonance spectrometry.” Talanta 147, 383-389.
Kawka, O. E.; Simoneit B. R. T. (1994) “Hydrothermal pyrolysis of organic matter in Guaymas Basin: I. Comparison of hydrocarbon distributions in subsurface sediments and seabed petroleums.” Org. Geochem. 22, 947-978.
Kelemen, S. R.; Afeworki, M.; Gorbaty, M. L.; Sansone, M.; Kiatek, P.; Walters, C. c.; Freund, H.; Siskin, M.; Bence, A. E.; Curry, D. J.; Solum, M.; Pugmire, R. J.; Vandenbroucke, M.; Leblond, M.; Behar, F. (2007) “Direct characterization of kerogen by X-ray and solid-state ¹³C nuclear magnetic resonance methods.” Energy Fuels 21, 1548-1561.
Keppler, F.; Harper, D. B.; Kalin, R. M.; Meier-Augenstein, W.; Farmer, N.; Davis, S.; Schmidt, H-L.; Brown, D. M.; Hamilton, J. T. G. (2007) “Stable hydrogen isotope ratios of lignin methoxyl groups as a paleoclimate proxy and constraint of the geographical origin of wood.” New Phytol. 176, 600-609.
Keppler, F.; Kalin, R. M.; Harper, D. B.; McRoberts, W. C.; Hamilton, J. T. G. (2004) “Carbon isotope anomaly in the major plant C1 pool and its global biogeochemical implications.” Biogeosciences 1, 123-131.
Kvenvolden, K. A.; Rapp, J. B.; Hostettler, F. D.; Morton, J. L.; King, J. D.; Claypool, G. E. (1986) “Petroleum associated with polymetallic sulfide in sediment from Gorda Ridge.” Science (80-) 234, 1231-1234.
Lewan, M. D. (1998) “Sulphur-radical control on petroleum formation rates.” Nature 391, 164-166.
Lewan, M. D.; Kotarba, M. J.; Wieclaw, D.; Piestrzynski, A. (2008) “Evaluating transition-metal catalysis in gas generation from the Permian Kupferschiefer by hydrous pyrolysis.” Geochim. Cosmochim. Acta 72, 4069-4093.
Li, Y.; Zhang, L.; Xiong, Y.; Gao, S.; Yu, Z.; Peng, P. (2018) “Determination of position-specific carbon isotope ratios of propane from natural gas.” Org. Geochem. 119, 11-21.
Liguo, G.; XIanming, X.; Tian, H.; Song, Z. (2009) “Distinguishing gases derived from oil cracking and kerogen maturation: Insights from laboratory pyrolysis experiments.” Org. Geochem. 40, 1074-1084.
Liu, C.; Liu, P.; McGovern, G. P.; Horita, J. (2019) “Molecular and intramolecular isotope geochemistry of natural gases from the Woodford Shale, Arkoma Basin, Oklahoma.” Geochim. Cosmochim. Acta 255, 188-204.
Liu, C.; McGovern, G.; Liu, P.; Horita, J. (2018) “Position-specific carbon and hydrogen isotopic compositions of propane from natural gases with quantitative NMR.” Chem. Geol. 491, 14-26.
Lloyd, M. K.; Eldridge, D. L.; Stolper, D. A. (2021) “Clumped 13CH₂D and 12CHD2 compositions of methyl groups from wood and synthetic monomers: Methods, experimental and theoretical calibrations, and initial results.” Geochim. Cosmochim. Acta 297, 233-275.
Lorant, F.; Prinzhofer, A.; Behar, F.; Huc, A-Y. (1998) “Carbon isotopic and molecular constraints on the formation and the expulsion of thermogenic hydrocarbon gases.” Chem. Geol. 147, 249-264.
Mango, F. D. (1992) “Transition metal catalysis in the generation of petroleum and natural gas.” Geochim. Cosmochim. Acta 56, 553-555.
Mango, F. D.; Hightower, J. W.; James, A. T. (1994) “Role of transition-metal catalysis in the formation of natural gas.” Nat. 1994 3686471 368, 536-538.
Mango, F. D.; Jarvie, D.; Herriman, E. (2009) “Natural gas at thermodynamic equilibrium implications for the origin of natural gas.” Geochem. Trans. 10, 1-12.
Milkov A. V.; Etiope G. (2018) “Revised genetic diagrams for natural gases based on a global dataset of >20,000 samples.” Org. Geochem. 125, 109-120.
Milkov A. V.; Faiz, M.; Etiope, G. (2020) “Geochemistry of shale gases from around the world: Composition, origins, isotope reversals and rollovers, and implications for the exploration of shale plays.” Org. Geochem. 143, 103997.
Monson, K. D.; Hayes, J. M. (1980) “Biosynthetic control of the natural abundance of carbon 13 at specific positions within fatty acids in Escherichia coli. Evidence regarding the coupling of fatty acid and phospholipid synthesis.” J. Biol. Chem. 255, 11435-11441.
Ni, Y.; Ma, Q.; Ellis, G. S. (2011) “Fundamental studies on kinetic isotope effect (KIE) of hydrogen isotope fractionation in natural gas systems.” Geochim. Cosmochim. Acta 75, 2696-2707.
Ni, Y.; Zhang, D.; Liao, F.; Gong, D.; Xue, P.; Yu, F.; Yu, J.; Chen, J.; Zhao, C.; Hu, J.; Jin, Y. (2015) “Stable hydrogen and carbon isotopic ratios of coal-derived gases from the Turpan-Hami Basin, NW China.” Int. J. Coal Geol. 152, 144-155.
Ono, S.; Wang, D. T.; Gruen, D. S.; Lollar, B. S.; Zahniser, M. S.; McManus, B. J.; Nelson, D. D. (2014) “Measurement of a doubly substituted methane isotopologue, ¹³CH₃D, by tunable infrared laser direct absorption spectroscopy.” Anal. Chem. 86, 6487-6494.
Pan, C.; Geng, A. I Yu, L. (2008) “Kerogen pyrolysis in the presence and absence of water and minerals. 1. Gas components.” Energy Fuels 22, 416-427.
Peterson, B. K.; Formolo, M. J.; Lawson, M. (2018) “Molecular and detailed isotopic structures of petroleum: Kinetic Monte Carlo analysis of alkane cracking.” Geochim. Cosmochim. Acta 243, 169-185.
Piasecki, A.; Sessions, A.; Lawson, M.; Ferreira, A. A.; Santos Neto, E. V. S.; Eiler, J. M. (2016) “Analysis of the site-specific carbon isotope composition of propane by gas source isotope ratio mass spectrometer.” Geochim. Cosmochim. Acta 188, 58-72.
Piasecki, A.; Sessions, A.; Lawson, M.; Ferreira, A. A.; Santos Neto, E. V.; Ellis, G. S.; Lewan, M. D.; Eiler, J. M. (2018) “Position-specific ¹³C distributions within propane from experiments and natural gas samples.” Geochim. Cosmochim. Acta 220, 110-124.
Price, L. C.; Schoell, M. (1995) “Constraints on the origins of hydrocarbon gas from compositions of gases at their site of origin.” Nat. 1995 3786555 378, 368-371.
Prinzhofer, A.; Girard, J-P.; Buschaert, S.; Huiban, Y.; Noirez, S. (2009) “Chemical and isotopic characterization of hydrocarbon gas traces in porewater of very low permeability rocks: The example of the Callovo-Oxfordian argillites of the eastern part of the Paris Basin.” Chem. Geol. 260, 269-277.
Ratkiewicz, A (2013) “First-principles kinetics of n-octyl radicals.” Prog. React. Kinet. Mech. 38, 323-341.
Ratkiewicz A . . . ; Bankiewicz, B.; Truong, T. N. (2010) “Kinetics of thermoneutral intermolecular hydrogen migration in alkyl radicals.” Phys. Chem. Chem. Phys. 12, 10988.
Ratkiewicz, A.; Truong, T. N. (2012) “Kinetics of the C—C bond beta scission reactions in alkyl radical reaction class.” J. Phys. Chem. A 116, 6643-6654.
Rodriguez, N. D.; Philp, R. P. (2010) “Geochemical characterization of gases from the Mississippian Barnett Shale, Fort Worth Basin, Texas.” Am. Assoc. Pet. Geol. Bull. 94, 1641-1656.
Rooney, M. A.; Claypool, G. E.; Chung, H. M. (1995) “Modeling thermogenic gas generation using carbon isotope ratios of natural gas hydrocarbons.” Chem. Geol. 126, 219-232.
Sadrameli, S. M. (2015) “Thermal/catalytic cracking of hydrocarbons for the production of olefins: A state-of-the-art review I: Thermal cracking review.” Fuel 140, 102-115.
Savage, P. E. (2000) “Mechanisms and kinetics models for hydrocarbon pyrolysis.” J. Anal. Appl. Pyrolysis 54, 109-126.
Saxby, J. D.; Riley, K. W. (1984) “Petroleum generation by laboratory-scale pyrolysis over six years simulating conditions in a subsiding basin.” Nat. 1984 3085955 308, 177-179.
Schoell, M. (1980) “The hydrogen and carbon isotopic composition of methane from natural gases of various origins.” Geochim. Cosmochim. Acta 44, 649-661.
Seewald, J. S. (2003) “Organic-inorganic interactions in petroleum producing sedimentary basins.” Nature 426, 327-333.
Seewald, J. S.; Benitez-Nelson, B. C.; Whelan, J. K. (1998) “Laboratory and theoretical constraints on the generation and composition of natural gas.” Geochim. Cosmochim. Acta 62, 1599-1617.
Sessions, A. L.; Sylva, S. P.; Summons, R. E.; Hayes, J. M. (2004) “Isotopic exchange of carbon-bound hydrogen over geologic timescales.” Geochim. Cosmochim. Acta 68, 1545-1559.
Shao, D.; Ellis, G. S.; Li, Y.; Zhang, T. (2018) “Experimental investigation of the role of rock fabric in gas generation and expulsion during thermal maturation: Anhydrous closed-system pyrolysis of a bitumen-rich Eagle Ford Shale.” Org. Geochem. 119, 22-35.
Sherwood, O. A.; Schwietzke, S.; Arling, V. A.; Etiope, G. (2017) “Global inventory of gas geochemistry data from fossil fuel, microbial and burning sources, version 2017.” Earth Syst. Sci. Data 9, 639-656.
Silverman, S.; Epstein, S. (1958) “Carbon isotopic compositions of petroleums and other sedimentary organic materials.” Am. Assoc. Pet. Geol. Bull. 42, 998-1012.
Sirjean, B.; Dames, E.; Wang, H.; Tsang, W. (2012) “Tunneling in hydrogen-transfer isomerization of n-alkyl radicals.” J. Phys. Chem. A 116, 319-332.
Stolper, D. A.; Sessions, A. L.; Ferreira, A. A.; Santos Neto E. V.; Schimmelmann, A; Shusta, S. S.; Valentine D. L.; Eiler, J. M. (2014) “Combined ¹³C-D and D-D clumping in methane: Methods and preliminary results.” Geochim. Cosmochim. Acta 126, 169-191.
Sung, H. C.; Brown, G. G.; White, R. R. (1945) “Thermal cracking of petroleum.” Ind. Eng. Chem. 37, 1153-1161.
Sweeney, J. J.; Burnham, A. K.; Braun, R. L. (1987) “Model of hydrocarbon generation from Type I Kerogen: Application to Uinta Basin, Utah.” Am. Assoc. Pet. Geol. Bull. 71, 967-985.
Tang, Y.; Huang, Y.; Ellis, G. S.; Wang, Y.; Kralert, P. G.; Gillaizeau, B.; Ma, Q.; Hwang, R. (2005) “A kinetic model for thermally induced hydrogen and carbon isotope fractionation of individual n-alkanes in crude oil.” Geochim. Cosmochim. Acta 69, 4505-4520.
Tang, Y.; Perry, J. K.; Jenden, P. D.; Schoell, M. (2000) “Mathematical modeling of stable carbon isotope ratios in natural gases.” Geochim. Cosmochim. Acta 64, 2673-2687.
Thiagarajan, N.; Kitchen, N.; Xie, H.; Ponton, C.; Lawson, M.; Formolo, M.; Eiler, J. (2020) “Identifying thermogenic and microbial methane in deep water Gulf of Mexico Reservoirs.” Geochim. Cosmochim. Acta, 275 (6964).
Thiagarajan, N.; Xie, H.; Ponton, C.; Eiler, J (2020) “Isotopic evidence for quasi-equilibrium chemistry in thermally mature natural gases.” Proc. Natl. Acad. Sci. 117, 3989-3995.
Tilley, B.; Muehlenbachs, K. (2013) “Isotope reversals and universal stages and trends of gas maturation in sealed, selfcontained petroleum systems.” Chem. Geol. 339, 194-204.
Tissot, B. P.; Welte, D. H. (1978) “Petroleum formation and occurrence.” Springer Berlin Heidelberg, Berlin, Heidelberg.
Ungerer, P. (1990) “State of the art of research in kinetic modelling of oil formation and expulsion.” Org. Geochem. 16, 1-25.
Ungerer, P.; Collell, J.; Yiannourakou, M. (2015) “Molecular modeling of the volumetric and thermodynamic properties of kerogen: Influence of organic type and maturity.” Energy Fuels 29, 91-105.
Vandenbroucke, M.; Largeau, C. (2007) “Kerogen origin, evolution and structure.” Org. Geochem. 38, 719-833.
Voter, A. F. (2007) “Introduction to the kinetic Monte Carlo method. In radiation effects in solids.” Springer Netherlands, Dordrecht, pp. 1-23.
Walters, C. C.; Freund, H.; Kelemen, S. R.; Peczak, P.; Curry, D. J. (2007) “Predicting oil and gas compositional yields via chemical structure-chemical yield modeling (CS-CYM): Part 2-Application under laboratory and geologic conditions.” Org. Geochem. 38, 306-322.
Waples, D. W.; Tornheim, L. (1978) “Mathematical models for petroleum-forming processes: carbon isotope fractionation.” Geochim. Cosmochim. Acta 42, 467-472.
Waples, D. W.; Tornheim, L. (1978) “Mathematical models for petroleum-forming processes: n-paraffins and isoprenoid hydrocarbons.” Geochim. Cosmochim. Acta 42, 457-465.
Wei, L.; Gao, Z.; Mastalerz, M.; Schimmelmann, A.; Gao, L.; Wang, X.; Liu, X.; Wang, Y.; Qiu, Z. (2019) “Influence of water hydrogen on the hydrogen stable isotope ratio of methane at low versus high temperatures of methanogenesis.” Org. Geochem. 128, 137-147.
Whiticar, M. J. (1996) “Stable isotope geochemistry of coals, humic kerogens and related natural gases.” Int. J. Coal Geol. 32, 191-215.
Wong, B. M.; Matheu, D. M.; Green, W. (2003) “Temperature and molecular size dependence of the high-pressure limit.” J. Phys. Chem. A 107, 6206-6211.
Wu, Y.; Zhang, Z.; Sun, L.; Li, Y.; Zhang, M.; Ji, L. (2019) “Stable isotope reversal and evolution of gas during the hydrous pyrolysis of continental kerogen in source rocks under supercritical conditions.” Int. J. Coal Geol. 205, 105-114.
Xia, X.; Gao, Y. (2019) “Kinetic clumped isotope fractionation during the thermal generation and hydrogen exchange of methane.” Geochim. Cosmochim. Acta 248, 252-273.
Xiao, Y. (2001) “Modeling the kinetics and mechanisms of petroleum and natural gas generation: A first principles approach.” Rev. Mineral. Geochem. 42, 382-436.
Xie, H.; Dong, G.; Formolo, M.; Lawson, M.; Liu, J.; Con, F.; Mangenot, X; Shuai, Y.; Ponton, C.; Eiler, J. (2021) “The evolution of intra- and inter-molecular isotope equilibria in natural gases with thermal maturation.” Geochim. Cosmochim. Acta 307, 22-41.
Xie, H.; Ponton, C.; Formolo, M. J.; Lawson, M.; Ellis, G. S.; Lewan, M. D.; Ferreira, A. A.; Morais, E. T.; Spigolon, A. L. D.; Sessions, A. L.; Eiler, J. M. (2020) “Position-specific distribution of hydrogen isotopes in natural propane: effects of thermal cracking, equilibration and biodegradation.” Geochim. Cosmochim. Acta 290, 235-256.
You, X.; Egolfopoulus, F. N.; Wang, H. (2009) “Detailed and simplified kinetic models of n-dodecane oxidation: The role of fuel cracking in aliphatic hydrocarbon combustion.” Proc. Combust. Inst. 32 (1), 403-410.
Young, E. D.; Rumble, D.; Freedman, P.; Mills, M. (2016) “A large-radius high-mass-resolution multiple-collector isotope ratio mass spectrometer for analysis of rare isotopologues of O₂, N₂, CH₄ and other gases.” Int. J. Mass Spectrometry, 401, 1-10.
Yuan, T.; Zhang, L.; Zhou, Z.; Xie, M.; Ye, L.; Qi, F. (2011) “Pyrolysis of n-heptane: Experimental and theoretical study.” J. Phys. Chem. A 115, 1593-1601.
Zhao, H.; Lin, C.; Larson, T. E.; McGovern, G. P.; Horita, J. (2020) “Bulk and position-specific isotope geochemistry of natural gases from the Late Cretaceous Eagle Ford Shale, south Texas.” Mar. Pet. Geol . . .
Zou, Y-R.; Cai, Y.; Zhang, C.; Zhang, X.; Peng, P. (2007) “Variations of natural gas carbon isotope-type curves and their interpretation-A case study.” Org. Geochem. 38, 1398-1415.

1. A method of evaluating an evolution of hydrocarbons from a reservoir, comprising:
a) obtaining one or more samples from one or more target hydrocarbon reservoirs over a period of time and assigning a time identifier and a location identifier to each of said samples;
b) fingerprinting each of said samples to obtain sample geochemical signatures including sample bulk composition signatures and sample carbon isotope signatures of one or more alkanes;
c) comparing said sample geochemical signatures to modeled signatures prepared by kinetic modeling to provide an assessment of i) a generation history for each of said samples, ii) a charge history for each of said reservoirs, and/or iii) a thermal maturity for each of said samples, wherein said kinetic modeling comprises:
i) devising a step-wise reaction network and providing an associated stoichiometry matrix that describes an evolution of said samples detailed to each carbon isotope isomer of each alkane, in which only one C—C bond cracks per each reaction step and a reaction rate constant is specific to a C—C bond defined by the molecule size, bond location, type of carbon (¹²C vs. ¹³C) at each end of said bond, and type of carbon immediately next to said C—C bond;
ii) building and solving coupled differential equations for said reaction network over time with a specified heating rate and an initial temperature to determine a concentration of each said carbon isotope isomer of each said alkane in said reaction network, by using 1) an Arrhenius equation to calculate a rate constant (k) for each reaction step, and 2) an initial reactant concentration (defined as relative abundances of different carbon isotope isomers for each alkane present at time 0) based on thermodynamic and/or statistical calculations and/or experimental measurements; and
iii) summing said concentration of each said carbon isotope isomer of each said alkane to provide said modelled signatures of each said carbon isotope isomer of each said alkane.

2. The method of claim 1, wherein said carbon isotope signatures are selected from isotope signatures of different petroleum fractions, compound specific isotopes of C1 up to C40, isotopologue specific isotope compositions of light hydrocarbons (C1-C4), and position specific isotope composition of light hydrocarbons.

3. The method of claim 1, wherein said fingerprinting step b) uses isotope ratio mass spectrometry (IRMS); GC isotope ratio MS (GC/IRMS); high resolution gas chromatography (HRGC); gas chromatography (GC); 2D gas chromatography (GCxGC), mass spectrometry (MS); gas chromatography-mass spectrometry (GC-MS); Fourier Transform Ion Cyclotron Resonance MS (FTICR-MS); thin layer chromatography (TLC); two dimensional TLC (2D TLC); capillary electrophoresis (CE); high pressure liquid chromatography (HPLC); Fourier Transform Infra-Red (FTIR) spectroscopy; X-ray fluorescence (XRF); atomic absorbance spectrophotometry (AAS); Inductively Coupled Plasma MS (ICP-MS); Ion Chromatography (IC); nuclear magnetic resonance (NMR); 2D GC-time of flight MS (GCxGC-TOFMS); saturate, aromatic, resin, and asphaltene levels (SARA levels); carbon, hydrogen, nitrogen, oxygen and sulfur analysis (CHNOS analysis); elemental analysis; or combinations thereof.

4. The method of claim 1, wherein said samples are selected from one or more of core samples; cutting samples; produced oil, water or gas samples; fractions of produced oil, water or gas samples; drilling mud samples; or mud gas samples.

5. The method of claim 1, wherein said location identifier includes depth and lateral placement (x, y and z coordinates).

6. The method of claim 1, further comprising comparing said sample signatures with said modelled signatures in order to i) correlate said samples to source rocks, ii) correlate said samples among said reservoirs, iii) elucidate a charge history of said samples, and/or iv) predict hydrocarbon properties in said reservoirs.

7. The method of claim 6, said method further comprising mapping said reservoir according to said reservoir charge history and said predicted hydrocarbon properties and said time identifier and said location identifier, and deciding a well placement plan based on said mapping.

8. The method of claim 6, further comprising:
a) using said predicted hydrocarbon properties in a reservoir model to predict production outcome;
b) optimizing a production plan based on said predicted production outcome; and
c) implementing said optimized production plan to produce hydrocarbons from said reservoirs.

9. The method of claim 8, wherein said reservoir model and said production plan includes one or more of well placement, well depth and lateral length, well arrangement, well completion, reservoir fracturing, reservoir stimulation, enhanced oil recovery techniques, and combinations thereof.

10. A kinetic method of modeling of hydrocarbons, comprising:
Step 1) constructing a reaction network describing an evolution of hydrocarbons;
Step 2) permuting all isotopic isomers for each species in said reaction network constructed in step 1, replacing each species with its permutation of isotopic isomers, then building out a larger reaction network via permuting reactants in the reaction network constructed in step 1;
Step 3) building a stoichiometry matrix for said larger reaction network constructed in step 2;
Step 4) calculating a rate constant for each reaction built out in step 2 with an Arrhenius equation in which a frequency factor and activation energy are a function of molecule size (number of carbon atoms), position of a reacting C—C bond, type of carbon (¹²C vs. ¹³C) of each carbon in the C—C bond, and type of carbon next to each end of the C—C bond (if any);
Step 5) building a differential equation system for said larger reaction network built in step 2, with stoichiometry built in step 3 and rate constants calculated in step 4;
Step 6) defining an initial condition for the differential equation system built in step 5 by estimating relative abundances of isotopic isomers via thermodynamic calculation or statistic calculation based on a lumped carbon isotope signature for all isomers (known as compound specific δ¹³C value for a given alkane) and thermodynamic settings, and augmenting calculations with available lab measurement data;
Step 7) defining a thermal history comprising initial temperature, temperature gradients and time span corresponding to a desired maturity (such as % R_{O}) changes;
Step 8) solving said differential equation system built in step 5 with initial conditions defined in step 6 over the thermal history defined in step 7, to generate a concentration of each species in said larger reaction network built in step 2 at each time step over the time span defined in step 7;
Step 9) preparing a model by processing results from step 8, comprising a sequence of nested grouping and summing all isotopic isomers of each alkane to obtain a hydrocarbon bulk composition, calculating compound specific carbon isotope signature based on a relative abundance of each isotopic isomer and a number of ¹³C substitution(s) within it, and calculating ratio(s) of isotopologue(s) and/or isotopomer(s) for each alkane;
Step 10) calibrating said model of step 9 with available geochemical analysis results of said samples from said reservoirs and refining steps 1-9 as needed to refine said model;
Step 11) using said model from step 10 to optimize and implement an exploration and/or production strategy.

11. The method of claim 10, further comprising implementing said exploration and/or production strategy to drill well(s) in said reservoirs.

12. The method of claim 11, further comprising implementing said exploration and/or production strategy to produce hydrocarbons from said well(s).

13. The method of claim 10, further comprising implementing said exploration and/or production strategy to produce hydrocarbons from said reservoirs.

14. The method of claim 10, wherein said hydrocarbons include compounds from C1 to C40 or larger.

15. The method of claim 10, wherein said isotopic isomers include compounds from C1 to C9.

16. The method of claim 10, wherein said isotopic isomers include compounds from C1 to C6.

17. The method of claim 10, wherein said isotopic isomers include compounds from C1 to C4.

18. The method of claim 10, wherein said isotopic isomers include carbon isotopes ¹²C and ¹³C.

19. The method of claim 10, wherein said hydrocarbons include compounds from C1 to C40 and said isotopic isomers include compounds from C1 to C4 and carbon isotopes ¹²C and ¹³C.

20. A method of optimizing hydrocarbon production, said method comprising:
a) modeling hydrocarbon bulk compositions and isotope compositions, wherein said bulk compositions are detailed to individual n-alkanes, where n is 1-41, and said isotope compositions are detailed to individual isotopomers within in each isotopologue of a given m-alkane where m is 1-3; and
b) using said modeling to optimize hydrocarbon production.
